(19) United States
(12) Patent Application Publication
(10) Pub. No.: US 2021/0231666 A1
(43) Pub. Date: Jul. 29, 2021

(54) CD33 MONOCYTES AS A BIOMARKER

(71) Applicants: Claire OLINGY, La Jolla, CA (US); Huy DINH, La Jolla, CA (US); LA JOLLA INSTITUTE FOR IMMUNOLOGY, La Jolla, CA (US)

(72) Inventors: Catherine HEDRICK, La Jolla, CA (US); Claire OLINGY, La Jolla, CA (US); Huy DINH, La Jolla, CA (US)

(73) Assignee: LA JOLLA INSTITUTE FOR IMMUNOLOGY, La Jolla, CA (US)

(21) Appl. No.: 15/734,974

(22) PCT Filed: Jun. 6, 2019
(86) PCT No.: PCT/US2019/035831

§ 371 Date: Dec. 3, 2020

Related U.S. Application Data
(60) Provisional application No. 62/681,560, filed on Jun. 6, 2018.

(51) Int. Cl. G01N 33/574

(52) U.S. Cl. CPC G01N 33/57423; G01N 2333/70578

(57) ABSTRACT

Provided herein are methods of determining whether a subject will respond to a treatment for a neoplasia, neoplastic disorder, tumor, cancer or malignancy, the method comprising detecting the presence or amount of CDS 3 expressing cells in a biological sample from the subject, comparing the measured presence or amount to a reference presence or amount, wherein a modified measured presence or amount as compared to the reference presence or amount is indicative that the subject will or will not respond to the treatment for a neoplasia, neoplastic disorder, tumor, cancer or malignancy. Also described herein are methods of treating a neoplasia, neoplastic disorder, tumor, cancer or malignancy in a subject, the method comprising modulating CDS 3 activity or expression in a myeloid cell, or alternatively, comprising administering a population of CD33hi monocytes or CD33hi macrophages.

### CROSS-REFERENCE TO RELATED APPLICATIONS

This application claims priority under 35 U.S.C. § 119(e) to U.S. Provisional Application No. 62/681,560, filed Jun. 6, 2018, the content of which is hereby incorporated by reference in its entirety.

This invention was made with support under grants R01 CA202987, P01 HL136275, and U01 CA224766 awarded by the National Institutes of Health. The government has certain rights in this invention.

### BACKGROUND

Lung cancer is the most common and deadly cancer type worldwide, accounting for nearly 1.8 million deaths in 2018¹. Immune checkpoint blockade (ICB), including anti-PD-1 monoclonal antibodies (aPD-1), produces positive clinical responses in approximately 30% of advanced non-small cell lung cancer (NSCLC) patients receiving aPD-1 monotherapy². Although aPD-1's primary mechanism of action is believed to be through reinvigoration of intratumoral antigen-experienced T cells³, monocytes and their macrophage/dendritic cell (DC) progeny are key regulators of the tumor immune microenvironment (TIME)⁴ and are reshaped by ICB^{5,6}.

Immunotherapies that broadly target monocytes/macrophages have displayed minimal success in early clinical trials, which may be due to the significant heterogeneity observed in these cell types and their ability to adopt both pro- and anti-tumor functions.

Thus, a need exists in the art to identify the cancers most suitably treated by these powerful new therapies. This disclosure satisfies this need and provides related advantages as well.

### SUMMARY OF THE DISCLOSURE

This summary is provided to introduce a selection of concepts in a simplified form that are further described below in the Detailed Description. This Summary is not intended to identify key aspects or essential aspects of the claimed subject matter.

The present disclosure aims to at least identify, characterize and/or isolate a population of cells that act as a biomarker for response to immunotherapy. Via the examination of the myeloid compartment in human cancer patients, the applicants have found that CD33 can be used as a predictive biomarker of patient response to immunotherapy.

The present disclosure relates broadly to a method of determining whether a subject will respond to a treatment for a neoplasia, neoplastic disorder, tumor, cancer or malignancy, the method comprising detecting the amount of CD33 in a biological sample from the subject, comparing the measured amount to a reference amount, wherein an increased measured amount compared to the reference amount is indicative that the subject will respond to the treatment for a neoplasia, neoplastic disorder, tumor, cancer or malignancy.

In certain aspects of the present disclosure, a method for determining whether a subject will respond to a treatment for a neoplasia, neoplastic disorder, tumor, cancer or malignancy is provided, the method comprising detecting the amount of CD33 in a biological sample from the subject, comparing the measured amount to a reference amount, wherein a decrease in the measured amount compared to the reference amount is indicative that the subject will not respond to the treatment for a neoplasia, neoplastic disorder, tumor, cancer or malignancy

In certain alternative aspects of the present disclosure, a method for determining whether a subject will respond to a treatment for a neoplasia, neoplastic disorder, tumor, cancer or malignancy is provided, the method comprising measuring the amount of CD33 in a biological sample from the subject, wherein a high amount of CD33 indicates that the subject will respond to a treatment for a neoplasia, neoplastic disorder, tumor, cancer or malignancy and a low amount of CD33 indicates that the subject will not respond to the treatment for a neoplasia, neoplastic disorder, tumor, cancer or malignancy.

In certain aspects of the present disclosure, a method for determining whether a subject will respond to a treatment for a neoplasia, neoplastic disorder, tumor, cancer or malignancy is provided, the method comprising detecting the amount of cells with high expression of CD33 in a biological sample from the subject, comparing the measured amount to a reference amount, wherein an increased measured amount compared to the reference amount is indicative that the subject will respond to the treatment for a neoplasia, neoplastic disorder, tumor, cancer or malignancy.

In other aspects of the present disclosure, a method for determining whether a subject will respond to a treatment for a neoplasia, neoplastic disorder, tumor, cancer or malignancy is provided, the method comprising measuring the presence or amount of cells with high expression of CD33 in a biological sample from the subject, wherein the presence or a high amount of cells with high expression of CD33 indicates that the subject will respond to the treatment for a neoplasia, neoplastic disorder, tumor, cancer or malignancy.

In alternative aspects of the present disclosure, a method of determining whether a subject will respond to a treatment for a neoplasia, neoplastic disorder, tumor, cancer or malignancy is provided, the method comprising detecting the amount of cells with low expression of CD33 in a biological sample from the subject, comparing the measured amount to a reference amount, wherein a decreased measured amount compared to the reference amount is indicative that the subject will not respond to the treatment for a neoplasia, neoplastic disorder, tumor, cancer or malignancy.

In certain aspects of the present disclosure, a method of determining whether a subject will respond to a treatment for a neoplasia, neoplastic disorder, tumor, cancer or malignancy is provided, the method comprising measuring the presence or amount of cells with low expression of CD33 in a biological sample from the subject, wherein the presence or a high amount of cells with low expression of CD33 indicates that the subject will not respond to the treatment for a neoplasia, neoplastic disorder, tumor, cancer or malignancy.

In certain other aspects of the present disclosure, the method comprises detecting or measuring the amount of CD33^{hi} myeloid cells in the biological sample. In alternative aspects of the present disclosure, the method comprises detecting CD33 monocytes or CD33^{hi} macrophages in the biological sample.

In certain aspects of the present disclosure, the method comprises detecting CD33^{hi}CD19−CD3−CD66b−CD56− cells or CD33^{hi}CD3−CD19−CD66b−CD56−HLA−DR+CD86+ cells in the biological sample.

In alternative embodiments, the method comprises detecting or measuring the amount of CD33^{hi} CD14^{dim}CD16⁺ monocytes or macrophages, CD33^{hi}CD14+CD16^{lo} monocytes or macrophages, CD33^{hi}CD14^{hi}CD16⁻ monocytes or macrophages, intermediate CD33^{hi}CD14⁺CD16⁺ monocytes or macrophages, and CD33^{hi}CD14^{lo}CD16⁺ monocytes or macrophages in the biological sample. In certain aspects of the present disclosure, the method comprises detecting CD33^{lo} myeloid cells in the biological sample. In alternative embodiments, the method comprises detecting CD33^{lo} monocytes or CD33^{lo} macrophages in the biological sample. In certain other aspects of the present disclosure, the method comprises detecting CD33^{lo}CD19⁻CD3⁻CD66b⁻CD56⁻ cells or CD33^{lo}CD3⁻CD19⁻CD66b⁻CD56−HLA⁻DR⁺CD86⁺ cells in the biological sample. In still other embodiments, the method comprises detecting CD33^{lo}CD14^{dim}CD16⁺ monocyte or macrophages, CD33^{lo}CD14⁺CD16^{lo} monocyte or macrophages, CD33^{lo}CD14^{hi}CD16⁻ monocytes or macrophages, CD33^{hi}CD14^{hi}CD16⁻ monocytes or macrophages, CD33^{lo}CD14^{hi}CD16⁻ monocytes or macrophages, intermediate CD33^{lo}CD14⁺CD16⁺ monocyte or macrophages, and/or CD33^{lo}CD14^{lo}CD16⁺ monocyte or macrophages in the biological sample.

In certain aspects of the present disclosure, the subject has a neoplasia, neoplastic disorder, tumor, cancer or malignancy. In certain other aspects, the subject has non-small cell lung cancer. In still other aspects, the subject has melanoma. In alterative aspects, the subject has breast cancer. In still other aspects, the subject has head and neck squamous cell carcinoma. In certain other aspects, the subject has renal cell carcinoma, bladder cancer, or Hodgkin's lymphoma.

In certain aspects of the present disclosure, wherein the biological sample is a blood sample. In alternative aspects of the present disclosure, the biological sample is taken from the subject prior to the treatment.

In certain aspects of the present disclosure, the treatment is a check point blocker. In some aspects of the present disclosure, the treatment is a PD-1 inhibitor or a PD-L1 inhibitor.

In some aspects of the present disclosure, the method is conducted prior to administration of the treatment to the subject in order to determine if the subject should receive the treatment.

In certain aspects of the present disclosure, the subject is a mammal. In alternative aspects, said subject is a human.

In certain aspects of the present disclosure, the use of a kit for sorting the disclosed cell populations from a biological sample is provided, where the kit is as defined herein.

Provided herein are methods of determining whether a subject will respond to a treatment for a neoplasia, neoplastic disorder, tumor, cancer or malignancy, the method comprising, or alternatively consisting essentially of, or yet further consisting of detecting the presence of or the amount of CD33 in a biological sample from the subject, comparing the measured amount to a reference amount, wherein a modified measured amount compared to the reference amount is indicative that the subject will or will not respond to the treatment for a neoplasia, neoplastic disorder, tumor, cancer or malignancy.

Further described herein are methods of determining whether a subject will respond to a treatment for a neoplasia, neoplastic disorder, tumor, cancer or malignancy, the method comprising, or alternatively consisting essentially of, or yet further consisting of detecting the presence of or amount of CD33 expressing cells in a biological sample from the subject, comparing the measured presence or amount to a reference presence or amount, wherein a modified measured presence or amount as compared to the reference presence or amount is indicative that the subject will or will not respond to the treatment for a neoplasia, neoplastic disorder, tumor, cancer or malignancy.

Further provided herein are methods of determining whether a subject will respond to a treatment for a neoplasia, neoplastic disorder, tumor, cancer or malignancy, the method comprising, or alternatively consisting essentially of, or yet further consisting of detecting the presence or measuring the amount of cells with high expression of CD33 in a biological sample from the subject, wherein the presence of cells with high expression of CD33 or a high amount of cells with high expression of CD33 indicates that the subject will respond to the treatment for a neoplasia, neoplastic disorder, tumor, cancer or malignancy.

Also described herein are methods of determining whether a subject will respond to a treatment for a neoplasia, neoplastic disorder, tumor, cancer or malignancy, the method comprising, or alternatively consisting essentially of, or yet further consisting of detecting the presence or measuring the amount of cells with low expression of CD33 in a biological sample from the subject, comparing the presence or measured amount to a reference amount, wherein a decreased presence or measured amount compared to the reference amount is indicative that the subject will not respond to the treatment for a neoplasia, neoplastic disorder, tumor, cancer or malignancy.

Also provided herein are kits comprising reagents to detect or measure the presence or amount of CD33 or CD33 expressing cells in a biological sample and instructions for use in the methods described herein. Also described herein are kits comprising, or alternatively consisting essentially of, or yet further consisting of reagents to detect or measure the amount of CD33^{hi} and/or CD³³ low expressing cells in a biological sample and instructions for use in the methods. In one aspect, the detecting or measuring is by a method comprising one or more of DNA microarrays, Real-time PCR, Chromatin immunoprecipitation (ChTP), flow cytometry, Western blotting, 2-D gel electrophoresis, immunoassays, or Fluorescence-activated cell sorting.

Furthermore, described herein are methods of treating a neoplasia, neoplastic disorder, tumor, cancer or malignancy in a subject, the method comprising, or alternatively consisting essentially of, or yet further consisting of modulating CD33 activity or expression in a myeloid cell. In one embodiment, the methods comprise, or alternatively consist essentially of, or yet further consist of administering an agent that modulates CD33 activity or expression. In one aspect, the agent is a small molecule, an antibody, a protein or a peptide. In another aspect, the agent is Azacitidine, Decitabine, or Lintuzumab.

Also provided herein are methods of treating a neoplasia, neoplastic disorder, tumor, cancer or malignancy in a subject, the method comprising, or alternatively consisting essentially of, or yet further consisting of administering a population of CD33^{hi} monocytes or CD33hi macrophages. In one aspect, the CD33 monocyte or macrophage population comprises one or more of: CD33hiCD19-CD3-CD66b-CD56− cells, CD33hiCD3-CD19-CD66b-CD56-HLA-DR+CD86+ cells, CD33hi CD14^{dim}CD16+ monocyte or macrophages, CD33hiCD14+CD16lo monocyte or macrophages, CD33^{hi}CD14^{hi}CD16⁻ monocytes or macrophages, CD33^{lo}CD14^{hi}CD16⁻ monocytes or macrophages, intermediate CD33^{hi}CD14⁺CD16⁺ monocyte or macrophages, and/or CD33hiCD14loCD16+ monocyte or macrophages in the biological sample.

In one particular aspect, the methods comprise, or alternatively consist essentially of, or yet further consist of modulating up CD33 activity or expression in a myeloid cell. Methods to lower expression include gene editing technologies such as interfering with RNA expression (administration of RNAi), CRISPR and TALEN. Methods to increase or enhance expression include transfection of monocytes, macrophages or precursors with exogenous DNA to increase expression of CD33 or activity, e.g. bone marrow transplantation of CD33high monocytes and/or macrophages. In a further aspect, the methods comprise, or alternatively consist essentially of, or yet further consist of modulating the amount of one or more of: CD33hiCD19−CD3−CD66b−CD56− cells, CD33hiCD3−CD19−CD66b−CD56−HLA−DR+CD86+ cells, CD33hi CD14^{dim}CD16+ monocyte or macrophages, CD33hiCD14+CD16lo monocyte or macrophages, intermediate CD33hiCD14+CD16+ monocyte or macrophages, and/or CD33hiCD14loCD16+ monocyte or macrophages, or CD33^{hi}CD14^{hi}CD16⁻ monocytes or macrophages in the biological sample. In another aspect, the methods comprise, or alternatively consist essentially of, or yet further consist of modulating down CD33 activity or expression in a myeloid cell. In a further aspect, the methods comprise, or alternatively consist essentially of, or yet further consist of modulating the amount of one or more of: CD33loCD19−CD3−CD66b−CD56− cells, CD33loCD3−CD19−CD66b−CD56−HLA-DR+CD86+ cells, CD33loCD14^{dim}CD16+ monocyte or macrophages, CD33loCD14+CD16lo monocyte or macrophages, intermediate CD33loCD14+CD16+ monocyte or macrophages, and/or CD33loCD14loCD16+ monocyte or macrophages in the biological sample.

All features of embodiments which are described in this disclosure and are not mutually exclusive can be combined with one another. Elements of one embodiment can be utilized in the other embodiments without further mention. Other aspects and features of the present disclosure will become apparent to those ordinarily skilled in the art upon review of the following description of specific embodiments in conjunction with the accompanying Figures.

### BRIEF DESCRIPTION OF THE DRAWINGS

A detailed description of specific embodiments is provided herein below with reference to the accompanying drawings in which:

FIG. 1 shows a non-limiting embodiment of a patient cohort, including clinical features of the cohort, used to determine whether peripheral blood monocytes are predictive of non-small cell lung carcinoma (NSCLC) patients' response to anti-PD-1 immunotherapy.

FIG. 2 shows a non-limiting embodiment of a mass cytometry (CyTof) panel.

FIG. 3 shows a non-limiting embodiment of mass cytometry of human peripheral blood mononuclear cells (PBMC).

FIG. 4 shows a non-limiting embodiment of plotted monocytes resulting in stratification based on responder status (e.g., responder or non-responder), both before and after immunotherapy.

FIG. 5 shows a non-limiting embodiment of a CD33lo cell population of myeloid cells (light grey) that is only present in non-responders.

FIG. 6 (top) shows, as non-limiting embodiment, response-predictive cells expressing myeloid markers. FIG. 6 (bottom) shows a non-limiting example of identified biaxial gates used to manually select response-predictive cells.

FIG. 7 shows a non-limiting embodiment of CD33lo monocytes present only in non-responders, and completely absent from responders both before and after the initiation of anti-PD-1 immunotherapy. This population increases in non-responders after the start of treatment, but does not change in responders. Healthy donors have primarily CD33hi monocytes.

FIG. 8 shows a non-limiting embodiment of CD33 presence in monocytes of responders, showing that CD33 expression is higher in classical (CD14+), nonclassical monocytes (CD14loCD16+), and intermediate monocytes (CD14+CD16+) of responders to checkpoint immunotherapy (e.g., anti-PD-1 therapy).

FIG. 9 shows a non-limiting embodiment of CD33 expression being globally higher on myeloid cells in responders.

FIG. 10 shows a non-limiting embodiment of CD33 expression in melanoma patients, showing that in patients with melanoma, CD33+ monocytes are increased in responders to checkpoint immunotherapy (e.g., anti-PD-1 therapy).

FIGS. 11A-11D shows CD33 is elevated in blood CD14+ monocytes of NSCLC patients responding to aPD-1. CellCNN identified cells elevated in NSCLC responders to aPD-1, circled (FIG. 11A top). Machine learning was used to identify cells that were able to predict patient response to anti-PD-1. This unbiased algorithm identified a population of monocytes with the phenotype shown below in the heatmap, characterized by high CD33 expression (FIG. 11A bottom). Biaxial contour plots of HLA-DR⁺Lineage⁻ monocytes (FIG. 11B) demonstrate a 2.6-fold increase in CD14⁺ monocyte CD33 expression of non-responders (NR) compared to responders (R) (FIG. 11C—Paired analysis of monocytes in blood samples from same patients (black line) at both pre- and post-anti-PD-1 therapy.) No differences in CD14⁺ or CD16⁺ frequency was detected (FIG. 11D) **p<0.01, Mann Whitney test.

FIG. 12A-12C show CD33 expression in healthy blood monocytes. FIG. 12A shows PBMCs from healthy donors were analyzed by flow cytometry for CD33 expression in CD14+HLA-DR⁺Lineage⁻ monocytes. FIG. 12B shows no correlation with age and FIG. 12C shows no correlation with gender was observed. Spearman correlation test.

FIGS. 13A-13B shows CD33 expression in the TIME. Co-culture of CD14+ monocytes and tumor cell lines (FIG. 13A) upregulates CD33. **p<0.05 by one-way ANOVA. Expression of CD33 in blood monocytes and tumor macrophages/DCs from early NSCLC patients is correlated (FIG. 13B) Data re-analyzed from¹¹. Spearman correlation test.

FIGS. 14A-14C: FIG. 14A shows B6 mice bearing CX3CR1hCD33/+ bone marrow (gray) or control mice (white) were subcutaneously implanted with (FIG. 14B) B16-F10 or (FIG. 14C) MC38 and treated with aPD-1 (black). Tumor growth was assessed using digital calipers.

FIG. 15 shows experimental design. Mass cytometry on peripheral blood samples collected from non-small cell lung cancer patients who were being treated with an anti-PD-1 immune checkpoint inhibitor, including either nivolumab or pembrolizumab was performed. Samples were obtained both before and after initiation of treatment. Clinical information as to whether the patient had responded to therapy was determined based on lack of disease progression within 6 months of treatment. Mass cytometry, which uses metal-labeled antibody detection by a time of flight mass spectrometer to simultaneously detect many different proteins on the single cell level was used. This technique can be used to measure around 40 proteins, which is only limited by the availability of rare earth metals of high enough purity.

FIG. 16 shows specialized CD33hi monocyte subsets are found in blood of responders to Anti-PD-1 immunotherapy. The circled cells are present in responder and absent in non-responder blood.

FIGS. 17 A-17B: FIG. 17A shows automated clustering of peripheral blood mononuclear cells (PBMCs) from NSCLC patients treated with anti-PD-1. FIG. 17B shows No difference in the overall frequency of leukocyte populations was detected in pre- vs. post-anti-PD-1 samples from non-responders or responders. Mann Whitney test.

FIGS. 18A-18B: FIG. 18A shows manual gating of CD14+ and CD16+ monocytes in peripheral blood mononuclear cells (PBMCs). FIG. 18B shows No difference in CD14+ or CD16⁺ frequency was detected. Mann Whitney test.

FIG. 19 shows CD33hi monocytes are present only in responders.

FIG. 20 shows CD33+ expression on monocytes predicts patient survival.

FIGS. 21A-21C: FIG. 21A shows co-culture of healthy CD14+ monocytes with tumor cells (MDA-MB-231 breast cancer cell line) to study response in CD33^{hi} vs. CD33^{lo} donors. FIG. 21B shows CD33^{hi} monocytes more robustly upregulate PD-L1 in co-culture with human cancer cells compared to CD33^{lo} monocytes. FIG. 21C shows CD33^{lo} monocytes more robustly upregulate CD206 in co-culture with human cancer cells compared to CD33^{hi} monocytes.

FIGS. 22A-22C show mouse model to study human CD33 biology. FIG. 22A shows human CD33 (hCD33) expression in peripheral blood of transgenic CX3CR1^{hCD33/+} mice and quantification of hCD33 in leukocyte subsets. FIG. 22B shows expression of hCD33 in control mice (left) vs. CX3CR1^{hCD33/+} mice (right). hCD33+ cells also express green fluorescence protein (gfp). FIG. 22C shows multi-lineage engraftment is the same in control (hCD33^{neg} mice) and hCD33+ mice, indicating that bone marrow transplantation of hCD33+ cells is an appropriate model for studying hCD33 function.

FIGS. 23A-23C: FIG. 23A shows analysis of PD-1 expression in tumor-draining lymph node (tdLN) T cells at day 13 in hCD33+ mice (B6 mice transplanted with CX3CR^{hCD33/+} bone marrow) (gray) or hCD33− control mice (white) bearing subcutaneous MC38 tumors. FIG. 23B shows ratio of naïve T cells (TN) to effector memory T cells (TEM) in tdLN. FIG. 23C shows MC38 tumor growth and response to anti-PD-1 (aPD-1) in hCD33− and hCD33+ mice. *p<0.05, Mann Whitney test.

In the drawings, embodiments are illustrated by way of example. It is to be expressly understood that the description and drawings are only for the purpose of illustrating certain embodiments and are an aid for understanding. They are not intended to be a definition of the limits of the disclosure.

### DETAILED DESCRIPTION

Illustrative embodiments of the disclosure will now be more particularly described. While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not delimit the scope of the disclosure.

The applicants have found that cancer patients (e.g., non-small cell lung carcinoma (NSCLC), head and neck squamous cell carcinoma HNSCC), breast cancer, and melanoma) being treated with checkpoint blockade immunotherapies, for example, PD-1 inhibitors or CTLA-4 inhibitors, have variable expression of CD33 within peripheral blood monocytes (CD3−CD19−CD66b−CD56−HLA−DR+CD86+ cells). Patients displaying high expression of CD33 on monocytes prior to initiation of immunotherapy treatment are more likely to be classified as responding to immunotherapy. The applicants also discovered found that patient blood can be examined prior to checkpoint blockade to determine whether the patient would respond to anti-PD-1 therapy by examining CD33 expression on monocytes.

Peripheral blood monocyte expression of CD33 can be used to predict cancer patient response to immunotherapy prior to the initiation of therapy or during therapy. This utilizes a less invasive blood draw compared to current molecular testing that requires a tumor biopsy. Additionally, the cell populations described herein can be used to stratify carcinoma patient populations for clinical trials or can be used as biomarkers when selecting therapeutic treatment.

### Definitions

Unless otherwise defined, all technical and scientific terms used herein have the same meaning as commonly understood by a person of ordinary skill in the art to which the present disclosure pertains. As used herein, and unless stated otherwise or required otherwise by context, each of the following terms shall have the definition set forth below.

In one embodiment, the methods described herein make use of the measured levels of the cell population of the present disclosure to detect surges, increases, or declines in cell numbers as predictive measures. As used herein, a “surge” or “increase” indicates a statistically significant increase in the level of relevant cells, typically from one measurement to one or more later measurements. In other instances, an increase in the level of relevant cells can be determined from one measure in a subject of interest relative to control (e.g., a value or a range of values for normal, i.e., healthy, individuals). Surges may be a two-fold increase in cell levels (i.e., a doubling of cell counts), a three-fold increase in cell levels (i.e., a tripling of cell numbers), a four-fold increase in cell levels (i.e., an increase by four times the number of cells in a previous measurement), or a five-fold or greater increase. In addition to the marked increase described as a surge, lesser increases in the levels of relevant cells may also have relevance to the methods of the present disclosure. Increases in cell levels may be described in terms of percentages. Surges may also be described in terms of percentages. For example, a surge or increase may be an increase in cell levels of 10%, 20%, 30%, 40%, 50%, 60%, 70%, 80%, 90%, or 100% or more. A “decline” indicates a decrease from one measurement to one or more later measurements. A decline may be a 5%, 10%, 15%, 20%, 25%, 30%, 35%, 40%, 45%, 50%, 55%, 60%, 65%, 70%, 75%, 80%, or 85% or greater decrease in cell levels from one measurement to one or more later measurements. In other instances, a decrease in the level of relevant cells can be determined from one measure in a subject of interest relative to control (e.g., a value or a range of values for normal, i.e., healthy, individuals).

In one embodiment, the surges, increases, or declines in cell numbers can be determined based on a comparison with a reference level derived from samples of at least 20 reference individuals without condition, a non-patient population. The surges or declines in cell numbers in a sample can also refer to a level that is elevated in comparison to the level of the cell numbers reached upon treatment, for example with an anti-cancer compound.

In one embodiment, the term “cancer” refers to a class of diseases in which a group of cells display uncontrolled growth, invasion, and/or metastasis. The term is meant to include, but not limited to, a cancer of the breast, respiratory tract, brain, reproductive organs, digestive tract, urinary tract, eye, liver, skin, head and neck, bone marrow, blood, thyroid, and parathyroid. The cancer may be a solid tumor, a non-solid tumor, or a distant metastasis of a tumor. Some specific examples of cancers include, but are not limited to, non-small cell lung cancer; small cell lung cancer; head and neck squamous cell carcinoma; renal cell carcinoma; bladder cancer; Hodgkin lymphoma; cutaneous squamous cell carcinoma; melanoma; myeloma; leukemia; Merkel-cell carcinoma; lymphomas; multiple myelomas; bone and connective tissue sarcomas; brain tumors; breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; gastric cancers; liver cancers; bladder cancers; gallbladder cancers; cholangiocarcinoma; lung cancers; testicular cancers; prostate cancers; penile cancers; oral cancers; basal cancers; salivary gland cancers; pharynx cancers; skin cancers; kidney cancers; melanomas or skin cancer, blood cancers such as myeloma, lymphoma, and Wilms' tumor. Examples of solid tumors include solid tumors of the breast, prostate, colon, pancreas, lung, gastric system, bladder, skin, and bone/connective tissue.

As used herein, “relapse” or “recurrence” may include the appearance of at least one new tumor lesion in, or new leukemia cells in the blood or bone marrow of, a subject who previously had cancer but has had no overt evidence of cancer as a result of surgery and/or therapy until relapse. Such recurrence of cancer cells can be local, occurring in the same area as one or more previous tumor lesions or leukemic cells, or distant, occurring in a previously lesion-free or cancer-cell free area, such as lymph nodes or other areas of the body.

As used herein, “response to treatment” may include complete response and partial response to treatment. A “complete response” (CR), in certain embodiments relating to e.g. cancer, is typically understood to include the disappearance of all target lesions and non-target lesions and normalization of tumor marker levels. A “partial response” (PR), in certain embodiments relating to cancer, is typically understood to include an at least 30% decrease in the sum of the diameters of target lesions. Generally speaking, in the context of embodiments relating to e.g. cancer, “response to treatment” may include an at least 30%-100% decrease in the sum of the diameters of target lesions, or disappearance of all target lesions and non-target lesions and normalization of tumor marker levels. “Progression” or “progressive disease” (PD), in certain embodiments relating to e.g. cancer, is typically understood to include an at least 20% increase in the sum of the diameters of target lesions, progression (increase in size) of any existing non-target lesions, and is also typically determined upon appearance of at least one new lesion. Non-CR/non-PD, in certain embodiments relating to e.g. cancer, is typically understood to include the persistence of one or more non-target lesions and/or maintenance of above-normal tumor marker levels. “Stable disease” (SD) is typically understood to include an insufficient increase to qualify for PD, but an insufficient decrease to qualify for PR. While the concepts of CR, PR, PD, and SD have been discussed in the context of cancer, the person of skill will readily understand that these concepts may also apply to other disease/conditions.

As used herein, the terms “treatment”, “treating”, and the like, may include amelioration or elimination of a developed disease or condition once it has been established or alleviation of the characteristic symptoms of such disease or condition. As used herein, these terms may also encompass, depending on the condition of the subject, preventing the onset of a disease or condition or of symptoms associated with the disease or condition, including for example reducing the severity of the disease or condition or symptoms associated therewith prior to affliction with the disease or condition. Such prevention or reduction prior to affliction may refer, in the context of cancer, to administration of at least one cancer therapeutic compound to a subject that is not at the time of administration afflicted with the disease or condition. “Preventing” may also encompass preventing the recurrence or relapse of a previously existing disease or condition or of symptoms associated therewith, for instance after a period of improvement.

As used herein, the phrase “respond to treatment” or similar phrases refer to the clinical benefit imparted to a patient suffering from a disease or condition, such as cancer, from or as a result of the treatment described herein. A clinical benefit includes a complete remission, a partial remission, a stable disease (without progression), progression-free survival, disease free survival, improvement in the time-to-progression (of the disease), improvement in the time-to-death, or improvement in the overall survival time of the patient from or as a result of the treatment described herein. There are criteria for determining a response to therapy and those criteria allow comparisons of the efficacy to alternative treatments (Slapak and Kufe, Principles of Cancer Therapy, in Harrisons's Principles of Internal Medicine, 13^{th} edition, eds. Isselbacher et al., McGraw-Hill, Inc., 1994). For example, a complete response or complete remission of cancer is the disappearance of all detectable malignant disease. A partial response or partial remission of cancer may be, for example, an approximately 50 percent decrease in the product of the greatest perpendicular diameters of one or more lesions or where there is not an increase in the size of any lesion or the appearance of new lesions.

The subject or patient can be any mammal, including a human. In particular, in the context of cancer, the subject can be a mammal who previously had cancer but appears to have recovered as a result of surgery and/or therapy, or who presently has cancer and is undergoing cancer therapy, or has completed a cancer therapeutic regime, or has received no cancer therapy. Non-limiting examples of mammals that can be assayed or treated include, murines, rats, canines, bovines, equines, felines, ovines, and human patients, adult and pediatric.

As used herein, the terms “therapeutically effective amount” and “effective amount” are used interchangeably to refer to an amount of a composition of the disclosure that is sufficient to result in the prevention of the development, recurrence, or onset of a disease or condition. For example, in certain embodiments e.g. cancer, these terms refer to an amount of a composition of the disclosure that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

In one non-limiting embodiment, the biological sample from the subject which is suspected of including cell populations described herein includes blood or a cell fraction thereof.

In one non-limiting embodiment, the biological sample from the subject which is suspected of including the cell population of the present disclosure includes blood, spleen, tumor tissue, bone marrow or a cell fraction thereof.

As used herein, a “cell fraction” of a biological sample may be obtained using routine clinical cell fractionation techniques, such as gentle centrifugation, e.g., centrifugation at about 300-800×g for about five to about ten minutes or fractionated by other standard methods.

In one non-limiting embodiment, the herein described sample can be obtained by any known technique, for example by drawing, by non-invasive techniques, or from sample collections or banks, etc.

In one non-limiting embodiment, the present disclosure provides a kit which includes reagents that may be useful for implementing at least some of the herein described methods. The herein described kit may include at least one detecting agent which is “packaged”. As used herein, the term “packaged” can refer to the use of a solid matrix or material such as glass, plastic, paper, fiber, foil and the like, capable of holding within fixed limits the at least one detection reagent. Thus, in one non-limiting embodiment, the kit may include the at least one detecting agent “packaged” in a glass vial used to contain microgram or milligram quantities of the at least one detecting agent. In another non-limiting embodiment, the kit may include the at least one detecting agent “packaged” in a microtiter plate well to which microgram quantities of the at least one detecting agent has been operatively affixed. In another non-limiting embodiment, the kit may include the at least one detecting agent coated on microparticles entrapped within a porous membrane or embedded in a test strip or dipstick, etc. In another non-limiting embodiment, the kit may include the at least one detecting agent directly coated onto a membrane, test strip or dipstick, etc. which contacts the sample fluid. Many other possibilities exist and will be readily recognized by those skilled in this art without departing from the disclosure. For example, the kit may include a combination of detecting agent which can be useful for cell sorting the cell populations of the present disclosure, as discussed elsewhere in the present document.

As used herein, a “purified cell population” refers to a cell population which has been processed so as to separate the cell population from other cell populations with which it is normally associated in its naturally occurring state. The purified cell population can, thus, represent an enriched cell population in that the relative concentration of the cell population in a sample can be increased following such processing in comparison to its natural state. In one embodiment, the purified cell population can refer to a cell population which is enriched in a composition in a relative amount of at least 80%, or at least 90%, or at least 95% or 100% in comparison to its natural state. Such purified cell population may, thus, represent a cell preparation which can be further processed so as to obtain commercially viable preparations. For example, in one embodiment, the cell preparation can be prepared for transportation or storage in a serum-based solution containing necessary additives (e.g., DMSO), which can then be stored or transported in a frozen form. In doing so, the person of skill will readily understand that the cell preparation is in a composition that includes a suitable carrier, which composition is significantly different from the natural occurring separate elements. For example, the serum-based preparation may comprise human serum or fetal bovine serum, which is a structural form that is markedly different from the form of the naturally occurring elements of the preparation. The resulting preparation includes cells that are in dormant state, for example, that may have slowed down or stopped intracellular metabolic reactions and/or that may have structural modifications to its cellular membranes. The resulting preparation includes cells that can, thus, be packaged or shipped while minimizing cell loss which would otherwise occur with the naturally occurring cells. A person skilled in the art would be able to determine a suitable preparation without departing from the present disclosure.

As used herein, the term “about” for example with respect to a value relating to a particular parameter (e.g. concentration, such as “about 100 mM”) relates to the variation, deviation or error (e.g. determined via statistical analysis) associated with a device or method used to measure the parameter. For example, in the case where the value of a parameter is based on a device or method which is capable of measuring the parameter with an error of 10%, “about” would encompass the range from less than 10% of the value to more than 10% of the value.

As used herein, the singular forms “a”, “an,” and “the” include plural referents unless the context clearly indicates otherwise.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein “a population of cells” intends a collection of more than one cell that is identical (clonal) or non-identical in phenotype and/or genotype.

As used herein, “substantially homogenous” population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers, phenotypic or genomic traits. In one aspect, the population is a clonal population.

As used herein, “heterogeneous” population of cells is a population having up to 69%, or alternatively up to 60%, or alternatively up to 50%, or alternatively up to 40%, or alternatively up to 30%, or alternatively up to 20%, or alternatively up to 10%, or alternatively up to 5%, or alternatively up to 4%, or alternatively up to 3%, or alternatively up to 2%, or alternatively up to 61%, or alternatively up to 0.5% identical phenotype, as measured by pre-selected markers, phenotypic or genomic traits.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, treatment excludes prophylaxis. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival (DFS), time to tumor recurrence (TTR), time to tumor progression (TTP), relative risk (RR), toxicity or side effects.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

The term “contacting” means direct or indirect binding or interaction between two or more entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “administer” and “administering” are used to mean introducing the therapeutic agent (e.g. polynucleotide, vector, cell, modified cell, population) into a subject. The therapeutic administration of this substance serves to attenuate any symptom, or prevent additional symptoms from arising. When administration is for the purposes of preventing or reducing the likelihood of developing cancer, the substance is provided in advance of any visible or detectable symptom or relapse. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound. The terms “upregulate” and “downregulate” and variations thereof when used in context of gene expression, respectively, refer to the increase and decrease of gene expression relative to a normal or expected threshold expression for cells, in general, or the sub-type of cell, in particular.

As used herein, the term “gene expression profile” refers to measuring the expression level of multiple genes to establish an expression profile for a particular sample.

As used herein, the term “reduce or eliminate expression and/or function of” refers to reducing or eliminating the transcription of said polynucleotides into mRNA, or alternatively reducing or eliminating the translation of said mRNA into peptides, polypeptides, or proteins, or reducing or eliminating the functioning of said peptides, polypeptides, or proteins. In a non-limiting example, the transcription of polynucleotides into mRNA is reduced to at least half of its normal level found in wild type cells.

As used herein, the term “increase expression of” refers to increasing the transcription of said polynucleotides into mRNA, or alternatively increasing the translation of said mRNA into peptides, polypeptides, or proteins, or increasing the functioning of said peptides, polypeptides, or proteins. In a non-limiting example, the transcription of polynucleotides into mRNA is increased to at least twice of its normal level found in wild type cells.

An “an effective amount” or “efficacious amount” is an amount sufficient to achieve the intended purpose, non-limiting examples of such include: initiation of the immune response, modulation of the immune response, suppression of an inflammatory response and modulation of T cell activity or T cell populations. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a human has or is suspected of having a cancer or neoplastic disorder.

A “composition” typically intends a combination of the active agent, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (I), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

As used herein, the term “animal” refers to living multi-cellular vertebrate organisms, a category that includes, for example, mammals and birds. The term “mammal” includes both human and non-human mammals, e.g., bovines, canines, felines, rat, murines, simians, equines and humans. Additional examples include adults, juveniles and infants.

As used herein, the term “antibody” (“Ab”) collectively refers to immunoglobulins (or “Ig”) or immunoglobulin-like molecules including but not limited to antibodies of the following isotypes: IgM, IgA, IgD, IgE, IgG, and combinations thereof. Immunoglobulin-like molecules include but are not limited to similar molecules produced during an immune response in a vertebrate, for example, in mammals such as humans, rats, goats, rabbits and mice, as well as non-mammalian species, such as shark immunoglobulins (see Feige, M. et al. Proc. Nat. Ac. Sci. 41(22): 8155-60 (2014)). Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997. One of skill in the art can monitor expression of mRNA using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

One of skill in the art can use methods such as RNA interference (RNAi), CRISPR, TALEN, ZFN or other methods that target specific sequences to reduce expression and/or increase expression/and or function of CD33.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “Cas9” refers to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the ¹2th and ¹3th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

The term “culturing” refers to growing cells in a culture medium under conditions that favor expansion and proliferation of the cell. The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

“Classical monocyte” intends a monocyte that is (CD14+).

“Nonclassical monocyte” intends a monocyte that is (CD14^{lo}CD16⁺).

“Intermediate monocyte” intends a monocyte that is (CD14⁺CD16⁺).

“Checkpoint inhibitor” intends a drug or therapy that blocks certain proteins made by some immune cells, such as T cells and some cancer cells. These proteins assist with immune responses, and keep immune responses in check. When these proteins are blocked, the brakes on the immune system are released and T cell are able to inhibit the growth or kill cancer cells. Non-limiting examples of checkpoint proteins on T cell or cancer cells include PD-1/PD-L1 and CTLA-4/B7-1/B7-2. Checkpoint inhibitors are used to treat cancer alone or in combination with other therapies and treatments. Non-limiting examples of PD-1 inhibits include Pembrolizumab (Keytruda), Nivolumab (Opdivo) and Cemiplimab (Libtayao), which have been shown to treat melanoma, NSCLC, kidney cancer, bladder cancer, head and neck cancers, and Hodgkin lymphoma. Non-limiting examples of PD-L1 inhibitors include Atezolizumab (Tecentriq), Avelumab (Bavencio), and Durvalumab (Imfinzil). These drugs have been shown to treat bladder cancer, NSCLC, Merkel cell skin cancer (Merkel cell carcinoma). Non-limiting examples of drugs that target CTLA-1 include Ipilimumab (Yervoy) which has been used to treat melanoma and other cancers. Additional treatments are under development as described for example in Darvin et al. (2018) Exper. & Mol. Med. 50, Article number 165 and Tang et al. (2018) Nature Reviews Drug Discover 17:854-855. The Cancer Research Institute reports that over 2,250 clinical trials are evaluating PD-1/L1 checkpoint inhibitors and 1,716 trials are assessing regimens that combine PD-1/L1 immune checkpoints with other therapies. 240 drug targets are being evaluated in the current landscape. See cancerresearch.org/news/2018/pd-1-11-checkpoint-inhibitor-landscape-analysis, last accessed Jun. 5, 2019.

The disclosure is further illustrated by the following non-limiting examples.

### Modes for Carrying Out the Disclosure

Lung cancer is the most common and deadly cancer type worldwide, accounting for 18.4% of total cancer-related deaths in 2018. Immune checkpoint blockade (ICB), including anti-PD-1 monoclonal antibodies (aPD-1), has been shown to produce positive clinical responses in approximately 30% of advanced non-small cell lung cancer (NSCLC) patients. Although aPD-1's primary mechanism of action is believed to be through reinvigoration of intratumoral antigen-experienced T cells, monocytes and their macrophage/dendritic cell progeny are key regulators of the tumor immune microenvironment and are reshaped by ICB. Immunotherapies that broadly target monocytes/macrophages have displayed minimal success in early clinical trials, which may be due to the significant heterogeneity observed in these cell types and their ability to adopt both pro- and anti-tumoral functions. Using high-dimensional mass cytometry, Applicants discovered that high CD33 expression is a feature of peripheral blood classical monocytes in NSCLC patients responding to aPD-1. Furthermore, Applicants found that transgenic mice expressing the human CD33 protein in monocyte lineage cells display faster tumor growth, but have more activated PD-1+ T cells and respond more robustly to aPD-1 compared to control mice lacking human CD33. Based on these findings, Applicants identified how CD33 expression in monocytes contributes to anti-tumoral immune responses, and assessed the therapeutic potential of CD33hi monocytes in increasing the efficacy of aPD-1-based immunotherapies in preclinical lung cancer models. The results of this work have provided novel insight into whether monocyte subsets can be leveraged to enhance response to ICB in lung cancer and improve understanding of how monocytes contribute to effective anti-tumoral immune responses.

Provided herein are methods of determining whether a subject will respond to a treatment for a neoplasia, neoplastic disorder, tumor, cancer or malignancy, the method comprising, or alternatively consisting essentially of, or yet further consisting of detecting the amount of CD33 in a biological sample from the subject, comparing the measured amount to a reference amount, wherein a modified measured amount compared to the reference amount is indicative that the subject will or will not respond to the treatment for a neoplasia, neoplastic disorder, tumor, cancer or malignancy. In one aspect, the modified measured amount is an increase in the measured amount. In a further aspect, the increase in the measured amount is indicative that the subject will respond to the treatment for a neoplasia, neoplastic disorder, tumor, cancer or malignancy. As used herein, “increase in the measured amount” refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold increase in the measured amount compared to the reference amount.

In one embodiment, the measured amount is CD33 high expression in the biological sample. As used herein, “CD33 high expression” or an increase in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold increase in CD33 expression compared to the reference amount. One of skill in the art can monitor expression of mRNA using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc. Cells sorting methods such as Fluorescence-activated cell sorting (FACS) can be employed to sort for and measure the desired cell populations. Several anti-CD33 antibodies are also available that can be employed to determine CD33 expression. Non-limiting examples of anti-CD333 antibodies are PE anti-human CD33 Antibody by Biolegend (Cat. No.: 303403), Anti-CD33 antibody ab203253 by Abcam, and CD33 Monoclonal Antibody (Cat. No.: 2B7C12) by Invitrogen.

In one embodiment, the CD33 high expressing cells comprise, or alternatively consist essentially of, or yet further consist of CD33^{hi} monocytes or CD33^{hi} macrophages in the biological sample. In a further embodiment, the methods comprise, or alternatively consist essentially of, or yet further consist of detecting or measuring the amount of CD33^{hi}CD19-CD3−CD66b−CD56− cells or CD33^{hi}CD3−CD19−CD66b−CD56−HLA−DR+CD86+ cells in the biological sample. In a separate embodiment, the methods comprise, or alternatively consist essentially of, or yet further consist of detecting or measuring the amount of CD33^{hi}CD14^{dim}CD16⁺ monocyte or macrophages, CD33^{hi}CD14^{hi}CD16⁻ monocytes or macrophages, CD33^{hi}CD14+CD16^{lo} monocyte or macrophages, intermediate CD33^{hi}CD14+CD16+ monocyte or macrophages, or CD33^{hi}CD14^{lo}CD16+ monocyte or macrophages in the biological sample.

In another aspect, the modified measured amount is a decrease in the measured amount. As used herein, “decrease in the measured amount” refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold decrease in the measured amount compared to the reference amount.

In a further aspect, the decrease in the measured amount compared to the reference amount is indicative that the subject will not respond to the treatment for a neoplasia, neoplastic disorder, tumor, cancer or malignancy. In one embodiment, the measured amount is CD33 low expression in the biological sample. As used herein, “CD33 low expression” or decrease in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold decrease in CD33 compared to the reference amount. In another embodiment, the measured amount is CD33 high expression in the biological sample. CD33 high expressing cells can comprise, or alternatively consist essentially of, or yet further consist of CD33^{hi} monocytes or CD33^{hi} macrophages in the biological sample. In a further embodiment, the methods comprise, or alternatively consist essentially of, or yet further consist of detecting or measuring the amount of CD33^{hi}CD19−CD3−CD66b−CD56− cells or CD33^{hi}CD3−CD19−CD66b−CD56-HLA-DR+CD86+ cells in the biological sample. In a separate embodiment, the methods comprise, or alternatively consist essentially of, or yet further consist of detecting or measuring the amount of CD33^{hi}CD14^{dim}CD16⁺ monocyte or macrophages, CD33^{hi}CD14+CD16^{lo} monocyte or macrophages, CD33^{hi}CD14^{hi}CD16⁻ monocytes or macrophages, intermediate CD33^{hi}CD14+CD16+ monocyte or macrophages, or CD33^{hi}CD14^{lo}CD16⁺ monocyte or macrophages in the biological sample.

Further described herein are methods of determining whether a subject will respond to a treatment for a neoplasia, neoplastic disorder, tumor, cancer or malignancy, the method comprising, or alternatively consisting essentially of, or yet further consisting of detecting the presence or amount of CD33 expressing cells in a biological sample from the subject, comparing the measured presence or amount to a reference presence or amount, wherein a modified measured presence or amount as compared to the reference presence or amount is indicative that the subject will or will not respond to the treatment for a neoplasia, neoplastic disorder, tumor, cancer or malignancy. In one aspect, the CD33 expressing cells measured are cells with high CD33 expression. As used herein, “CD33 high expression” or an increase in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold increase in CD33 expression compared to the reference amount. In a further aspect, the measured presence or amount of cells with high CD33 expression is indicative that the subject will respond to the treatment for a neoplasia, neoplastic disorder, tumor, cancer or malignancy. In one embodiment, the CD33 high expressing cells comprise CD33 monocytes or CD33^{hi} macrophages in the biological sample. In a further embodiment, the methods comprise, or alternatively consist essentially of, or yet further consist of detecting or measuring the amount of CD33^{hi}CD19−CD3−CD66b−CD56− cells or CD33^{hi}CD3−CD19−CD66b−CD56-HLA-DR+CD86+ cells in the biological sample. In a separate embodiment, the methods comprise, or alternatively consist essentially of, or yet further consist of detecting or measuring the amount of CD33^{hi} CD14^{dim}CD16⁺ monocyte or macrophages, CD33^{hi}CD14+CD16^{lo} monocyte or macrophages, intermediate CD33^{hi}CD14+CD16⁺ monocyte or macrophages, or CD33^{hi}CD14^{lo}CD16⁺ monocyte or macrophages in the biological sample. In another aspect, the CD33 expressing cells measured are cells with low CD33 expression.

In a further aspect, the measured presence or amount of cells with low CD33 expression is indicative that the subject will not respond to the treatment for a neoplasia, neoplastic disorder, tumor, cancer or malignancy. As used herein, “CD33 low expression” or decrease in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold decrease in CD33 compared to the reference amount.

Further provided herein are methods of determining whether a subject will respond to a treatment for a neoplasia, neoplastic disorder, tumor, cancer or malignancy, the method comprising, or alternatively consisting essentially of, or yet further consisting of detecting the presence or measuring the amount of cells with high expression of CD33 in a biological sample from the subject, wherein the presence of cells with high expression of CD33 or a high amount of cells with high expression of CD33 indicates that the subject will respond to the treatment for a neoplasia, neoplastic disorder, tumor, cancer or malignancy. In one aspect, the methods comprise, or alternatively consist essentially of, or yet further consist of detecting the presence of or measuring the amount of CD33^{hi} myeloid cells in the biological sample. In a further aspect, the methods comprise, or alternatively consist essentially of, or yet further consist of detecting the presence or measuring the amount of classical or non-classical monocytes in the biological sample. Classical monocytes refer to CD14+CD16lo monocytes, intermediate monocytes refer to CD14+CD16+ monocytes, and non-classical monocytes refer to CD14loCD16+ monocytes.

In one embodiment, the CD33 high expressing cells comprise CD33 monocytes or CD33 macrophages in the biological sample. As used herein, “CD33 high expression” or an increase in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold increase in CD33 expression compared to the reference amount.

In a further embodiment, the methods comprise, or alternatively consist essentially of, or yet further consist of detecting or measuring the amount of CD33^{hi}CD19−CD3−CD66b-CD56− cells or CD33^{hi}CD3−CD19−CD66b−CD56-HLA-DR+CD86+ cells in the biological sample. In a separate embodiment, the methods comprise, or alternatively consist essentially of, or yet further consist of detecting or measuring the amount of CD33^{hi} CD14^{dim}CD16+ monocyte or macrophages, CD33^{hi}CD14+CD16^{lo} monocyte or macrophages, CD33^{hi}CD14^{hi}CD16⁻ monocytes or macrophages intermediate CD33^{hi}CD14+CD16+ monocyte or macrophages, or CD33^{hi}CD14^{lo}CD16⁺ monocyte or macrophages in the biological sample. In a separate aspect, the methods comprise, or alternatively consist essentially of, or yet further consist of detecting the presence or measuring the amount of classical monocytes in the biological sample. In a further aspect, the methods comprise, or alternatively consist essentially of, or yet further consist of detecting the presence or measuring the amount of non-classical monocytes in the biological sample.

Also described herein are methods of determining whether a subject will respond to a treatment for a neoplasia, neoplastic disorder, tumor, cancer or malignancy, the method comprising, or alternatively consisting essentially of, or yet further consisting of detecting the presence or measuring the amount of cells with low expression of CD33 in a biological sample from the subject, comparing the presence or measured amount to a reference amount, wherein a decreased presence or measured amount compared to the reference amount is indicative that the subject will not respond to the treatment for a neoplasia, neoplastic disorder, tumor, cancer or malignancy. In one aspect, the methods comprise, or alternatively consist essentially of, or yet further consist of detecting the presence or measuring the amount of CD33^{lo} myeloid cells in the biological sample. As used herein, “CD33 low expression” or decrease in CD33 expression refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold decrease in CD33 compared to the reference amount.

In another aspect, the methods comprise, or alternatively consist essentially of, or yet further consist of detecting the presence or measuring the amount of classical or non-classical monocytes in the biological sample. In one embodiment, the methods comprise, or alternatively consist essentially of, or yet further consist of detecting the presence or measuring the amount of classical monocytes in the biological sample. In another embodiment, the methods comprise, or alternatively consist essentially of, or yet further consist of detecting the presence or measuring the amount of non-classical monocytes in the biological sample. Classical monocytes refer to CD14+CD16lo monocytes, intermediate monocytes refer to CD14+CD16+ monocytes, and non-classical monocytes refer to CD14loCD16+ monocytes.

The methods can comprise, or alternatively consist essentially of, or yet further consist of detecting the presence of or measuring the amount of CD33^{lo} monocytes or CD33^{lo} macrophages in the biological sample. In a separate embodiment, the methods comprise, or alternatively consist essentially of, or yet further consist of detecting the presence of or amount of CD33^{lo}CD19−CD3−CD66b−CD56− cells or CD33^{lo}CD3−CD19−CD66b−CD56-HLA-DR+CD86+ cells in the biological sample. In one aspect, methods comprise, or alternatively consist essentially of, or yet further consist of detecting the presence of or amount of CD33^{lo}CD14^{dim}CD16⁺ monocyte or macrophages, CD33^{lo}CD14+CD16^{lo} monocyte or macrophages, CD33^{lo}CD14^{hi}CD16⁻ monocytes or macrophages, intermediate CD33^{lo}CD14+CD16⁺ monocyte or macrophages, or CD33^{lo}CD14^{lo}CD16⁺ monocyte or macrophages in the biological sample.

In one aspect, for any of the methods described herein, the subject has a neoplasia, neoplastic disorder, tumor, cancer or malignancy. In a further aspect, the cancer is a carcinoma or a leukemia. In one particular aspect, the subject has non-small cell lung cancer or melanoma. In yet another aspect, the subject is suffering from a cancer of the group of: Stage I, Stage II, Stage III or Stage IV.

In one aspect, for any of the methods described herein, the biological sample comprises, or alternatively consists essentially of, or yet further consists of a blood sample. In another aspect, the biological sample is taken from the subject prior to the treatment. In yet another aspect, the methods described herein further comprise, or alternatively consist essentially of, or yet further consist of taking a further biological sample from the subject after treatment to monitor treatment efficacy. Ina further aspect, the methods can further comprise, or alternatively consist essentially of, or yet further consist of administering an effective amount of the same treatment or different therapy to the subject. In one embodiment, administering an effective amount of the same treatment to the subject comprises, or alternatively consists essentially of, or yet further consists of administering a modified effective amount of the treatment. In one aspect, a modified effective amount is lower than an effective amount dose. The modified effective amount can be about 1.5 fold, about 2 fold, about 3 fold, about 4 fold, about 5 fold, about 6 fold, about 7 fold, about 8 fold, about 9 fold, about 10 fold lower than the effective amount. In another aspect, the modified effective amount is higher than an effective amount dose. The modified effective amount can be about 1.5 fold, about 2 fold, about 3 fold, about 4 fold, about 5 fold, about 6 fold, about 7 fold, about 8 fold, about 9 fold, about 10 fold higher than the effective amount.

In one embodiment, for the methods described herein, a positive response to treatment for the neoplasia, neoplastic disorder, tumor, cancer or malignancy comprises, or alternatively consists essentially of, or yet further consists of one or more of an increase in survival time; an increase in elongation in time to tumor progression; a reduction in tumor mass; a reduction in tumor burden and/or a prolongation in time to tumor metastasis; elongated time to tumor recurrence, tumor response, complete response, partial response, stable disease, progressive disease; an increase in progression free survival, or an increase in overall survival. In another embodiment, the treatment is selected from the group of: a first line treatment, a second line treatment, a third line treatment or a fourth line treatment. In a further aspect, the treatment comprises, or alternatively consists essentially of, or yet further consists of a check point blocker.

In one particular embodiment, the treatment comprises, or alternatively consists essentially of, or yet further consists of a PD-1 inhibitor or a PD-L1 inhibitor. In one aspect, the check point blocker is selected from the group of: an anti-PDL-1 antibody, an anti-PD-1 antibody, Nivolumab, Pembrolizumab, Cemiplimab, Atezolizumab, Avelumab, Darvalumab, or an equivalent thereof. In another aspect, the PD-1 inhibitor is selected from the group of an anti-PD-1 antibody, Nivolumab, Pembrolizumab, Cemiplimab, or an equivalent thereof. In a distinct aspect, the PD-L1 inhibitor is selected from the group of Atezolizumab, Avelumab, Darvalumab, or an equivalent thereof. For the methods treatment comprising, or alternatively consisting essentially of, or yet further consisting of a PD-1 inhibitor, the cancer is from the group of: melanoma, squamous non-small cell lung cancer, renal cell carcinoma, myeloma, cutaneous squamous cell carcinoma, bladder cancer, Hodgkin's lymphoma, a unresectable or metastatic solid tumor, or small cell lung cancer. For the methods treatment comprising, or alternatively consisting essentially of, or yet further consisting of a PDL-1 inhibitor, the cancer is from the group of: bladder cancer, non-small cell lung cancer, small cell lung cancer, or Merkel-cell carcinoma.

In a further embodiment, the methods described herein can further comprise, or alternatively consist essentially of, or yet further consist of administering an effective amount of the treatment to the subject where the subject has been determined to respond to the treatment. In another embodiment, the methods described herein can further comprise, or alternatively consist essentially of, or yet further consist of administering an effective amount of the treatment to the subject where the subject has been determined to not respond to the treatment. The methods can further comprise, or alternatively consist essentially of, or yet further consist of administering an effective amount of the treatment to the subject where the subject has been determined to respond to the treatment. In one aspect, the methods can further comprise, or alternatively consist essentially of, or yet further consist of administering an effective amount of an expanded population of CD33^{high} expressing cells to the subject where the subject has been determined to not to respond to the treatment. In one aspect, the CD33^{high} expressing cells further comprise, or alternatively consist essentially of, or yet further consist of CD33^{hi}CD19−CD3−CD66b−CD56− cells or CD33^{hi}CD3−CD19−CD66b−CD56-HLA-DR+CD86+ cells. In another aspect, the CD33^{high} expressing cells further comprise, or alternatively consist essentially of, or yet further consist of CD33^{hi} CD14^{dim}CD16⁺ monocyte or macrophages, CD33^{hi}CD14+CD16^{lo} monocyte or macrophages, CD33^{hi}CD14^{hi}CD16-monocytes or macrophages, intermediate CD33^{hi}CD14+CD16⁺ monocyte or macrophages, and CD33^{hi}CD14^{lo}CD16⁺ monocyte or macrophages. The methods can further comprise, or alternatively consist essentially of, or yet further consist of administering an effective amount of the treatment to the subject. In one aspect, the treatment is selected from the group of: a first line treatment, a second line treatment, a third line treatment or a fourth line treatment. In another aspect, the treatment is co-administered with an effective amount of a second therapy. In a further aspect, the second therapy is administered prior to, concurrently or subsequently to the treatment. The second therapy can comprise, or alternatively consist essentially of, or yet further consist of one or more of surgical rescission, radiation therapy, light therapy, or a chemotherapy. In one particular embodiment, the effective amount of the second therapy is a low dose therapy. A low dose therapy can refer to a therapy in which lower than the effective amount of a drug is administered.

For any of the methods described herein, the subject can be a mammal. In one aspect, the mammal is of the group of: a canine, a feline, an equine, a bovine, an ovine, a murine, a rat, a simian or a human patient. In another aspect, the subject is a female. In a different aspect, the subject is a male. In yet another aspect, the subject is a pediatric patient.

Further described herein are compositions comprising, or alternatively consisting essentially of, or yet further consisting of one or more of: an expanded population of CD33^{high} expressing cells, CD33^{hi}CD19−CD3−CD66b−CD56− cells, CD33^{hi}CD3−CD19−CD66b−CD56− HLA-DR+CD86+ cells, CD33^{hi} CD14^{dim}CD16⁺ monocyte or macrophages, CD33^{hi}CD14+CD16^{lo} monocyte or macrophages, CD33^{hi}CD14^{hi}CD16⁻ monocytes or macrophages, CD33^{lo}CD14^{hi}CD16⁻ monocytes or macrophages, CD33^{hi}CD14^{hi}CD16⁻ monocytes or macrophages intermediate CD33^{hi}CD14+CD16⁺ monocyte or macrophages, and/or CD33^{hi}CD14^{lo}CD16⁺ monocyte or macrophages and a pharmaceutically acceptable carrier. In another embodiment, compositions comprising, or alternatively consisting essentially of, or yet further consisting of one or more of: CD33loCD19−CD3−CD66b−CD56− cells, CD33loCD3−CD19−CD66b−CD56-HLA-DR+CD86+ cells, CD33loCD14^{dim}CD16+ monocyte or macrophages, CD33loCD14+CD16lo monocyte or macrophages, CD33^{lo}CD14^{hi}CD16⁻ monocytes or macrophages, intermediate CD33loCD14+CD16+ monocyte or macrophages, and/or CD33loCD14loCD16+ monocyte or macrophages and a pharmaceutically acceptable carrier are also described. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for monocytes or macrophages, or will be able to ascertain such, using routine experimentation. Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

Also provided herein are kits comprising reagents to detect or measure the presence or amount of CD33 or CD33 expressing cells in a biological sample and instructions for use in the methods described herein. Also described herein are kits comprising, or alternatively consisting essentially of, or yet further consisting of reagents to detect or measure the amount of CD33^{hi} and/or CD³³low expressing cells in a biological sample and instructions for use in the methods. In one aspect, the detecting or measuring is by a method comprising one or more of DNA microarrays, Real-time PCR, Chromatin immunoprecipitation (ChTP), flow cytometry, Western blotting, 2-D gel electrophoresis, immunoassays, or Fluorescence-activated cell sorting. The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit. As amenable, these suggested kit components may be packaged in a manner customary for use by those of skill in the art. For example, these suggested kit components may be provided in solution or as a liquid dispersion or the like.

Furthermore, described herein are methods of treating a neoplasia, neoplastic disorder, tumor, cancer or malignancy in a subject, the method comprising, or alternatively consisting essentially of, or yet further consisting of modulating CD33 activity or expression in a myeloid cell. In one embodiment, the methods comprise, or alternatively consist essentially of, or yet further consist of administering an agent that modulates CD33 activity or expression. In one aspect, the agent is a small molecule, an antibody, a protein or a peptide. In another aspect, the agent is azacitidine, decitabine, or lintuzumab.

Also provided herein are methods of treating a neoplasia, neoplastic disorder, tumor, cancer or malignancy in a subject, the method comprising, or alternatively consisting essentially of, or yet further consisting of administering a population of CD33hi monocytes or CD33hi macrophages. In one aspect, the CD33hi monocyte or macrophage population comprises one or more of: CD33hiCD19−CD3−CD66b−CD56− cells, CD33hiCD3−CD19−CD66b−CD56−HLA−DR+CD86+ cells, CD33hi CD14^{dim}CD16⁺ monocyte or macrophages, CD33hiCD14+CD16lo monocyte or macrophages, CD33^{hi}CD14^{hi}CD16⁻ monocytes or macrophages intermediate CD33hiCD14+CD16⁺ monocyte or macrophages, and/or CD33hiCD14loCD16⁺ monocyte or macrophages in the biological sample.

In one particular aspect, the methods comprise, or alternatively consist essentially of, or yet further consist of modulating up CD33 activity or expression in a myeloid cell. As used herein, “modulating up CD33 activity or expression” refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold increase in CD33 expression. In a further aspect, the methods comprise, or alternatively consist essentially of, or yet further consist of modulating the amount of one or more of: CD33hiCD19-CD3−CD66b−CD56− cells, CD33hiCD3−CD19−CD66b−CD56-HLA-DR+CD86+ cells, CD33hi CD14^{dim}CD16⁺ monocyte or macrophages, CD33hiCD14+CD16lo monocyte or macrophages, CD33^{hi}CD14^{hi}CD16⁻ monocytes or macrophages intermediate CD33hiCD14+CD16⁺ monocyte or macrophages, and/or CD33hiCD14loCD16⁺ monocyte or macrophages in the biological sample. In another aspect, the methods comprise, or alternatively consist essentially of, or yet further consist of modulating down CD33 activity or expression in a myeloid cell. As used herein, “modulating down CD33 activity or expression” refers to about 1.5 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold or about 100 fold decrease in CD33 expression. In a further aspect, the methods comprise, or alternatively consist essentially of, or yet further consist of modulating the amount of one or more of: CD33loCD19−CD3−CD66b−CD56− cells, CD33loCD3−CD19-CD66b−CD56-HLA-DR+CD86+ cells, CD33loCD14^{dim}CD16⁺ monocyte or macrophages, CD33loCD14+CD16lo monocyte or macrophages, CD33^{lo}CD14^{hi}CD16⁻ monocytes or macrophages, intermediate CD33loCD14+CD16⁺ monocyte or macrophages, and/or CD33loCD14loCD16⁺ monocyte or macrophages in the biological sample.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

If the patient is identified as not likely to respond to immune checkpoint inhibitor therapy, another therapy can be utilized, that include but are not limited to The term “chemotherapy” encompasses cancer therapies that employ chemical or biological agents or other therapies, such as radiation therapies, e.g., a small molecule drug or a large molecule, such as antibodies, RNAi and gene therapies. Non-limiting examples of chemotherapies are provided below and include topoisomerase inhibitors, pyrimidine antimetabolites, anti-metabolites, and cisplatin drugs.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

A therapy comprising a pyrimidine antimetabolite includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFOX6, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Bevacizumab (BV) is sold under the trade name Avastin® by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis. Bevacizumab is also in the class of cancer drugs that inhibit angiogenesis (angiogenesis inhibitors).

Trifluridine/tipiracil (CAS Number 733030-01-8) is sold under the trade name of Lonsurf. It is a combination of two active pharmaceutical ingredients: trifluridine, a nucleoside analog, and tipiracil hydrochloride, a thymidine phosphorylase inhibitor. Trifluridine has the chemical formula C₁₀H₁₁F₃N₂O₅ and is also known as α,α,α-trifluorothymidine; 5-trifluromethyl-2′-deoxyuridine; and FTD5-trifluoro-2′-deoxythymidine (CAS number 70-00-8). Tipiracil has the chemical formula C₉H₁₁ClN₄O₂ and inhibits the enzyme thymidine phosphorylase, preventing rapid metabolism of trifluridine, increasing the bioavailability of trifluridine. Equivalents of trifluridine/tipiracil include trifluridine alone, trifluridine that modified to increase its halflife and/or resistance to metabolism by thymidine phosphorylase, or substitution of one or both of trifluridine and/or tipiracil hydrochloride with a chemical equivalent. Non-limiting examples of chemical equivalents include pharmaceutically acceptable salts or solvates of the active ingredients.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin.

Oxaliplatin (trans-/-diaminocyclohexane oxalatoplatinum; L-OHP; CAS No. 61825-94-3) is sold under the trade name of Elotaxin. It is a platinum derivative that causes cell cytotoxicity. Oxaliplatin forms both inter- and intra-strand cross links in DNA, which prevent DNA replication and transcription, causing cell death. Non-limiting examples of an equivalent of oxaliplatin include carboplatin and cisplatin.

If the patient is, then the checkpoint inhibitor therapy can be used as a monotherapy or in combination with another therapy concurrently or sequentially.

The following examples describe some exemplary modes of making and practicing embodiments of the disclosure. It should be understood that these examples are for illustrative purposes only and are not meant to limit the scope of the compositions and methods described herein.

### EXAMPLES

### General Methodologies

In the methods noted below, the following methods were used, unless otherwise specifically noted.

Citrus and CellCNN are machine learning algorithms referenced as: CelCNN (Arvaniti, E. & Claassen, M. Sensitive detection of rare disease-associated cell subsets via representation learning. Nature Communications 2017 8:1 8, 14825 (2017)) and Citrus (Bruggner R V, et al Automated identification of stratifying signatures in cellular subpopulations. Proc Natl Acad Sci USA. 2014; 111(26):E2770-7. doi: 10.1073/pnas.1408792111. PubMed PMID: 24979804; PMCID: PMC4084463.)

Mass cytometry data analysis and CyTOF data normalization. Data was normalized (including EQ bead-removal) using Matlab-based normalization software NormalizerR2013a_Win64.

For CyTOF data analysis, all gating and t-SNE was performed using Cytobank analysis software (Cytobank Inc.). Before downstream analysis, CD45+ Lin^{lo} (CD3+CD19+CD66b+) cells were manually gated. For clustering analysis, total monocytes were defined by gating on a Lin^{lo} t-SNE map in order to exclude only the Lin^{hi} cells and enrich the monocytes knowing that some lineage markers are lowly expressed on monocytes. The resulting FCS files and XNL (gating) files were imported into the R (v3.4.1) and Bioconductor (v3.6) environment using the flowCore package (v1.40.3) (Hahne F, et al. flowCore: a Bioconductor package for high throughput flow cytometry. BMC Bioinformatics. 2009; 10:106.) All monocytes from 12 healthy individuals were pooled as recommended by the CyTOF workflow (Nowicka M, Krieg C, Weber L M, Hartmann F J, Guglietta S, Becher B, Levesque M P, Robinson Md. CyTOF workflow: differential discovery in high-throughput high-dimensional cytometry datasets. F1000Res. 2017; 6:748.) using the cells within the monocyte gate. Protein expression data from CyTOF was normalized using an inverse hyperbolic sine (arcsinh) transformation with a cofactor of 5. Clustering was performed based on Self-Organizing-Map method using the FlowSOM R package (v1.4.0) (Van Gassen S, et al. FlowSOM: Using self-organizing maps for visualization and interpretation of cytometry data. Cytometry A. 2015; 87(7):636-645.)

In total 227,371 monocytes were used to study human monocyte heterogeneity. Applicants ran FlowSOM clustering with a 100 random starting seeds and chose the solution from the run with the maximal mean of cluster correlation score compared to all other runs. The cluster correlation score for 2 runs was computed using a pairing strategy as follows: the first pair of clusters was determined using a Pearson correlation coefficient (PCC), the next one was chosen similarly using the remaining clusters until all pairs were determined. The final correlation score is the mean of PCC of all these pairs. Applicants used the delta area plot (ConsensusClusteringPlus R packagev1.43.0) (Wilkerson M D, Hayes D N. ConsensusClusterPlus: a class discovery tool with confidence assessments and item tracking. Bioinformatics. 2010; 26(12):1572-1573.) to evaluate the cluster robustness gained with the number of clusters from 2-20.

To exclude any individual variability and assure the reproducibility of the inferred clusters, Applicants tested the cluster correlation score of the final 8 clusters after leaving out 1 sample (12 cases) or 2 samples (132 cases) and found that it is highly reproducible. Visualization of mass cytometry data was performed using ggplot (v2.2.1).

Applicants also used the PhenoGraph algorithm (Levine J H, et al. Data-Driven Phenotypic Dissection of AML Reveals Progenitor-like Cells that Correlate with Prognosis. Cell. 2015; 162(1):184-197.), a clustering method based on k-nearest neighbor algorithm, implemented in the Cytofkit package (v1.6.1)8 with k=50 (Chen H, et al. Cytofkit: A Bioconductor Package for an Integrated Mass Cytometry Data Analysis Pipeline. PLoS Comput Biol. 2016; 12(9):e1005112).

Only clusters with a frequency of at least 1% in at least 3 donors were included in further analysis. Meta-clustering on the remaining clusters was applied using hierarchical clustering (hclust function in R with ‘complete’ agglomeration method and Euclidean distance) on the original clusters inferred by Phenograph.

Blood preparation. Peripheral blood mononuclear cells (PBMCs) were isolated from 10 mL freshly drawn heparinized blood of humans using Ficoll-Paque™ PLUS (GE Healthcare Biosciences AB) and SepMate™-50 (Stemcell Technologies Inc) according to the manufacturer's protocol. As a blocking step, cells were resuspended in 1 mL FACS buffer (1 mM EDTA, 1% BSA, 1% Human AB serum, 0.1% NaN3 in 1×PBS) and counted on the Accuri C6 (BD Biosciences). PBMCs were obtained from patients, frozen in FBS and 10% DMSO, stored in liquid nitrogen.

Monocyte staining for CyTOF analysis. First, cells were washed with 10 mL PBS, resuspended at 1×10⁷ cells/mL in PBS and stained with 1 mL 5 mM Cisplatin 195Pt (Fluidigm) for 5 min at room temperature to exclude dead cells. Thereafter, the cisplatin was quenched with Cell Staining Buffer (CSB; 2 mM EDTA, 0.1% BSA, 0.05% NaN3 in 1×PBS; 5× the volume of the cell suspension). Cells were then washed twice with 1 mL CSB and stained with 100 μL of heavy-metal isotope labeled antibody cocktail (Table S1) on ice. After 30 min, cells were washed twice with 1 mL CSB and fixed overnight at 4° C. with 100 μL 2% PFA (Electron Microscopy Sciences). Following fix, cells were washed twice with CSB and permeabilized with 100 μL of 1× Permeabilization buffer (eBiosciences) for 45 min on ice. Cells were washed twice with CSB and incubated with 100 μL 125 nM nucleic acid Ir-Intercalator (Fluidigm) for 30 min at room temperature. Finally, cells were washed twice with CSB, counted on Accuri C6 and washed thrice with MilliQ water. Cells were left pelleted until ready to run.

Immediately before acquisition on CyTOF 2 Helios (DVS Sciences/Fluidigm), cell pellets were resuspended at 1×106 cells/mL in 0.1×EQTM Four Element Calibration Beads (Fluidigm) and filtered into FACS tubes with cell strainer caps (Fisher Scientific). Quality control and tuning were performed on a daily basis before acquisition. After acquisition, files were automatically transformed into an fcs-format by the Helios Software (Fluidigm).

Antibody conjugation for CyTOF. For mass cytometry stainings, already conjugated antibodies were purchased from Fluidigm and purified antibodies were ordered from the companies listed in Table S1. Antibody labeling was performed with the Maxpar X8 Multi-Metal Labeling Kit (Fluidigm) according to the manufacturer's instructions. Conjugations were verified using BD CompBead Plus (BD Biosciences) and antibodies were titrated before use.

Human monocyte isolation. Human PBMC will be obtained as described above and monocytes (HLA-DR⁺Lineage⁻) will be sorted using fluorescence-activated cell sorting (FACS). CD33^{hi}CD14+CD16^{lo} and CD33^{lo}CD14+CD16^{lo} monocytes, intermediate CD33^{hi}CD14+CD16+ and CD33^{lo}CD14+CD16+ monocytes, or CD33^{hi}CD14^{lo}CD16+ and CD33^{lo}CD14^{lo}CD16+ monocytes and CD33^{hi}CD14^{hi}CD16⁻ monocytes and CD33^{lo}CD14^{hi}CD16-monocytes will be sorted from frozen PBMC or fresh blood as needed.

Expansion of CD33 cell populations. Monocytes for adaptive cell transfer can be produced in vitro from CD34+ cells (e.g. mononuclear cells) from bone marrow-derived monocytic cells (BMMs) or bone marrow-derived hematopoietic stem cells (HSCs) collected from bone marrow or cord blood using methods known in the art (Stec et al. (2007) J. Leukocyte Biology 82:594-602). For example, CD34+ cells are first expanded for 3-10 days in standard medium supplemented with Fetal Calf Serum (FCS) or an equivalent, stem cell factor (SCF), thrombopoietin (TPO) and Flt-3 Ligand (Flt-3L), and then differentiated to monocyte populations (e.g. CD14+CD16−) in IMDM medium supplemented with FCS, SCF, Flt-3L, IL-3 and M-CSF for 7-14 days. Varying the concentrations of SCF, Flt-3L and Vitamin D3 shift the ratio of monocyte subsets (Stec et al., (2007) supra.). In an alternative method, CD34+ cells are proliferated in serum-free conditions supplemented with SCF, 100 ng/ml Flt-3, 20 ng/ml IL-6, 20 ng/ml IL-3, in humidified atmosphere at 37° C. in 5% C02. The non-adherent cells are then collected and plated at 100,000 cells/cm2 in the presence of low endotoxin serum and 10 ng/ml MCSF for differentiation into monocyte subsets as described in Magga et al. (2012) J. Cell. Mol. Med. 16(5):1060-1073. These ex vivo methods of expanding monocytes are further captured in U.S. Pat. No. 8,574,903B2.

### Example 1

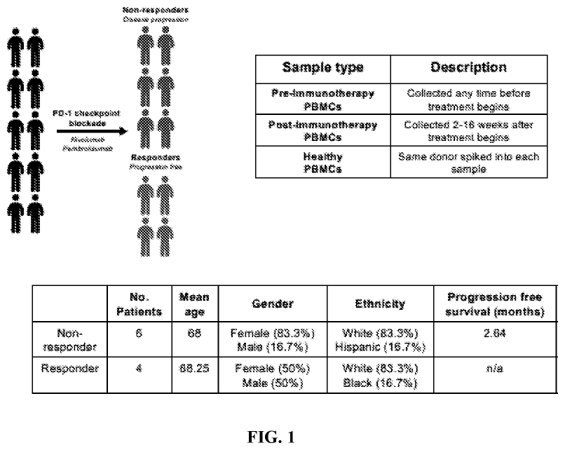

In this example, the applicants demonstrate the sample collection and patient metadata of a non-limiting example of a patient cohort used to predict patient response to immunotherapy. The applicants looked to determine whether the phenotype of peripheral blood monocytes can predict whether a NSCLC patient will respond to anti-PD-1 immunotherapy. Blood was collected from healthy donors and patients with cancer. (FIG. 1).

### Example 2

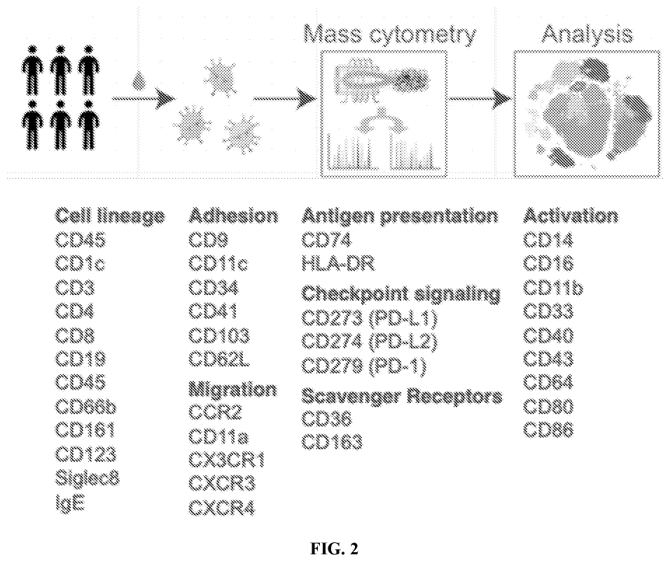

In this example, the applicants demonstrate the design of a CyTof panel for human PBMC's used to determine whether the phenotype of peripheral blood monocytes, using CyTOF mass cytometry markers (found on myeloid cells) can predict whether a NSCLC patient will respond to anti-PD-1 immunotherapy (FIG. 2)

### Example 3

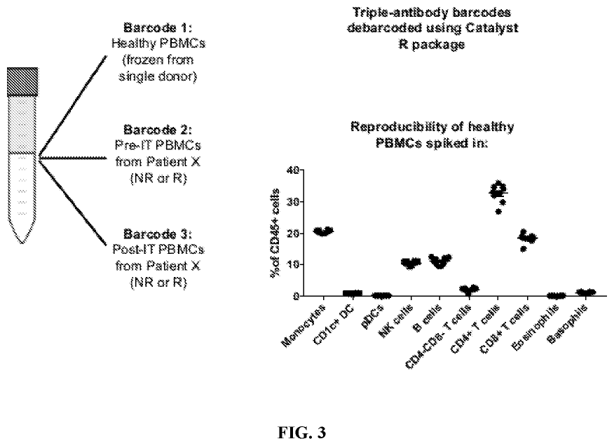

In this example, the applicants demonstrate the barcoding of the PBMC's described in Examples 1 and 2 with live cell palladium-conjugated CD45 antibodies, stained together. In each barcoded tube, the applicants combined healthy PBMC's from the same donor. (FIG. 3)

### Example 4

In this example, the applicants demonstrate the stratification of responders and non-responders using mass cytometry-based immunophenotyping of PBMC described in Examples 1-3. Applicants found that the patients stratify based on responder status before or after immunotherapy. (FIG. 4)

### Example 5

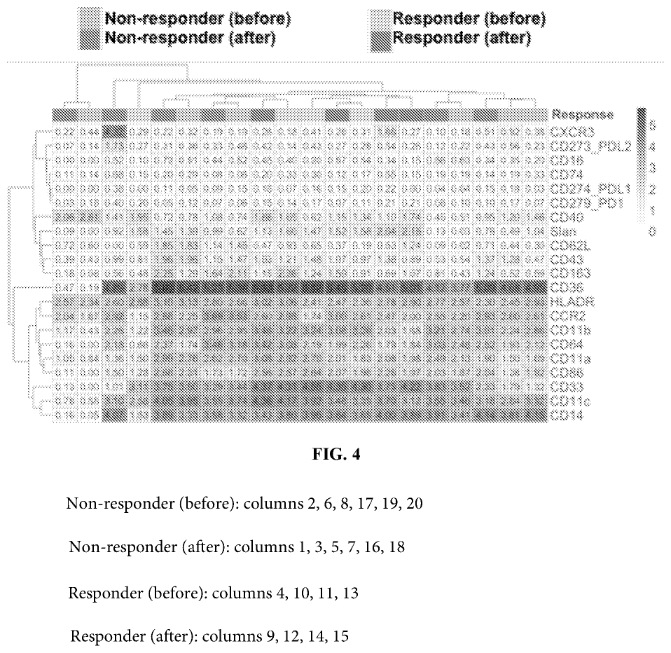

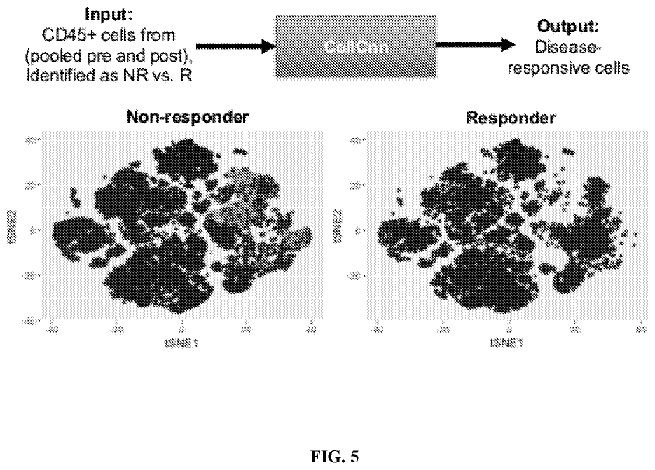

In this example, the applicants demonstrate the use of the CellCnn algorithm to identify a cell population predictive of whether NSCLC patients are responsive to PD-1 immunotherapy. All CD45+ cells were input into the CellCnn algorithm. A population of CD33lo myeloid cells (light grey) was found to only be present in non-responders. (FIG. 5)

### Example 6

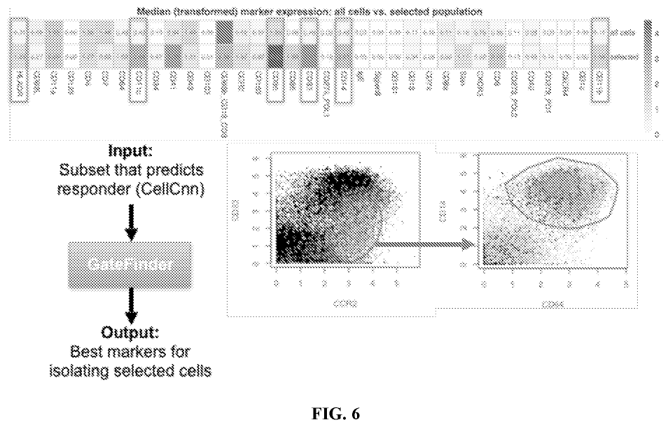

In this example, the applicants demonstrate the identification of responder-associated cells, finding that response-predictive cells express myeloid markers including HLADR, CD11c, CD36, CD33, CD13, Slan, and CD11b (FIG. 6—top). GateFinder was used to identify biaxial gates that could be used to manually select response-predictive cells identified by CellCnn.

### Example 7

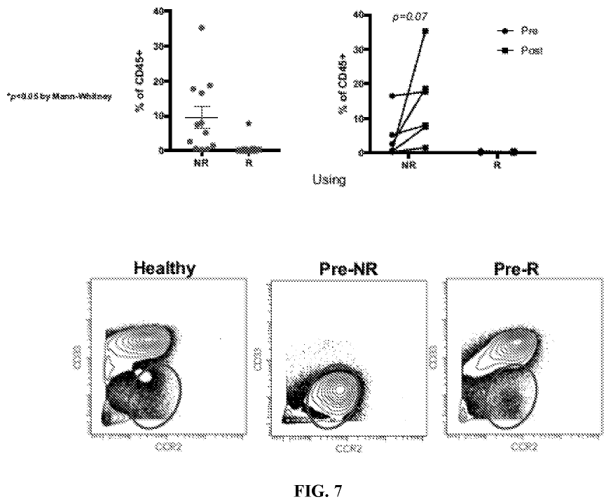

In this example, the applicants demonstrate that immunotherapy response-predictive cells can be identified by biaxial gating. Using biaxial gates identified by GateFinder and in FIG. 7 (top), the applicants found that CD33lo monocytes were only present in non-responders (NR), and completely absent from responders (R) both before and after the initiation of anti-PD-1 immunotherapy. Thus, the pre-therapy monocyte phenotype is sufficient for predicting immunotherapy response. Additionally, this population increases in non-responders after the start of treatment (FIG. 7—top right) but does not change in responders. Healthy donors have primarily CD33hi monocytes (FIG. 7—bottom). The cells were primarily identified using CD33, CCR2, and a second gate of CD14 vs. CD64.

### Example 8

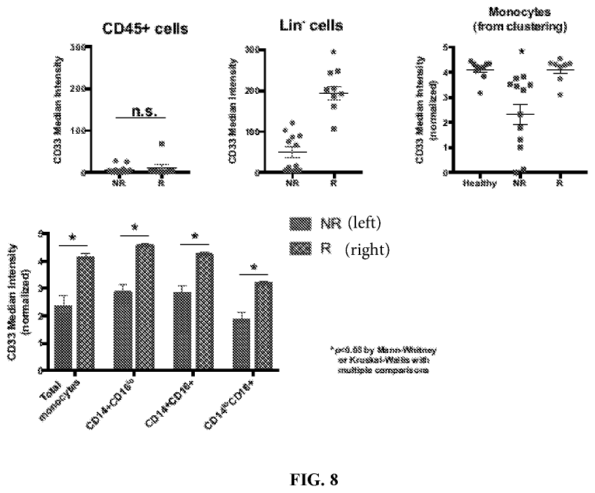

In this example, the applicants demonstrate that there is no difference in CD33 expression was seen in total CD45+ cells; however, Lin− cells (CD19−CD3−CD66b−CD56-) showed increased expression of CD33, as did monocytes identified through high-dimensional clustering with flowsom. The applicants have shown that CD33 expression is higher in all monocyte subsets of responders to checkpoint immunotherapy (e.g., anti-PD-1 therapy), including classical CD14+CD16lo, intermediate CD14+CD16+, and non-classical CD14loCD16+ monocytes. (FIG. 8)

### Example 9

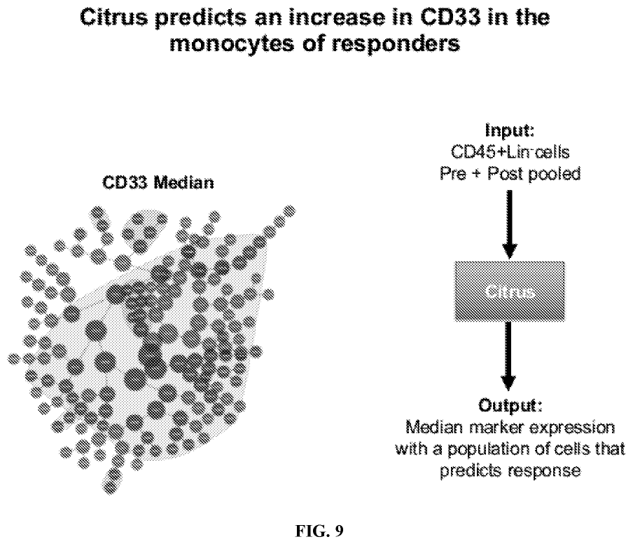

In this example, the applicants demonstrate the use of Citrus, a complementary algorithm for identifying disease-predictive cells, to identify CD33 expression as being globally higher on myeloid cells in responders. (FIG. 9)

### Example 10

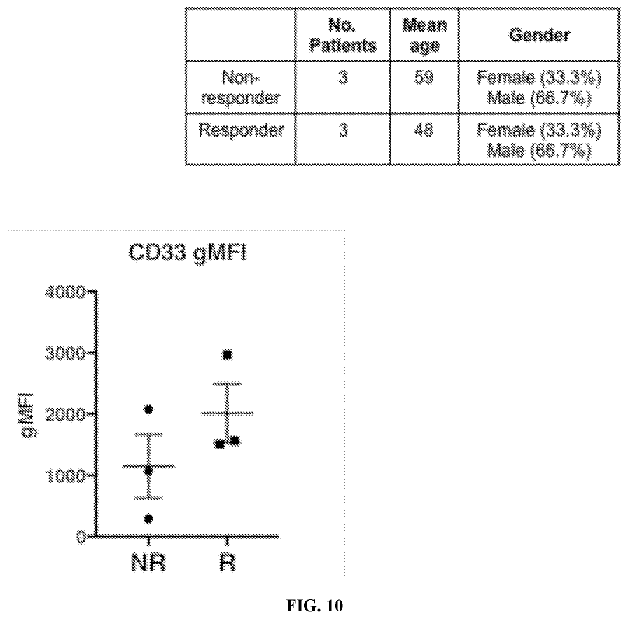

In this example, the applicants demonstrate a trend of increased in CD33 expression in melanoma patients undergoing treatment with immunotherapy in responders vs. non-responders. (FIG. 10)

### Example 11

In this example, the applicants demonstrate additional features and embodiments of the present disclosure.

Monocytes are a major source of intratumoral myeloid cells and can display both pro- and anti-tumoral functions^{7,8}. While classical monocytes (CD14⁺CD16⁻ in humans) promote tumorigenesis in murine cancer models^{7,9} and are associated with poor patient prognosis in multiple cancer types⁴, they are specifically elevated in melanoma patients positively responding to aPD-1¹⁰. Non-classical monocytes (CD14^{lo}CD16⁺ in humans) are anti-metastatic⁸ and excluded from early stage NSCLC tumors¹¹. Immunotherapy approaches targeting monocytes/macrophages such as CSF1R and CCR2 inhibitors have sought to broadly inhibit their accumulation in tumors, but have displayed minimal success in early phase clinical trials¹². It has been reported by Dr. Hedrick's laboratory¹³ and by others¹⁴ that human peripheral blood monocytes (including classical, intermediate, and non-classical monocytes) can be further discriminated into novel subsets.

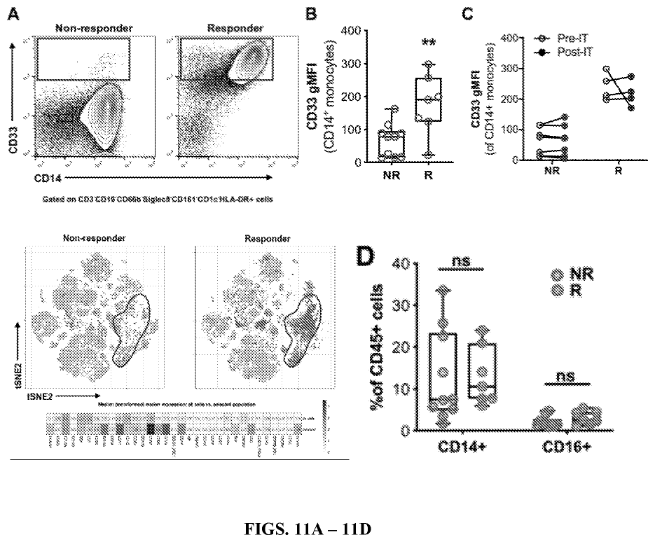

Using mass cytometry by time-of-flight (CyTOF), blood monocytes collected from NSCLC patients prior to treatment with aPD-1, including Nivolumab or Pembrolizumab were immunophenotyped. Peripheral blood mononuclear cells (PBMCs) collected from 17 stage III/IV NSCLC patients (10 non-responders, 7 responders) prior to aPD-1 monotherapy were analyzed by CyTOF. Non-responders were comprised of 2 males and 8 females with an average age of 69.9 years and average progression-free survival (PFS) of 2.3 months, while responders were comprised of 2 males and 5 females with an average age of 68.0 years and average PFS of 20.5 months. Using CellCNN, a supervised machine learning approach, it was discovered that high CD33 expression is a feature of CD14+ monocytes in NSCLC patients with positive clinical responses to aPD-1 (FIG. 11A). Manual gating of CD14+ monocytes (FIG. 11B) confirmed that CD33 expression is 2.6-fold higher in responders than non-responders (FIG. 11C), showing that CD33+ monocytes present in a cancer patient's blood before treatment can be used to predict positive response to immunotherapy, including anti-PD-1. Interestingly, both clustering and manual gating detected no differences in CD14⁺ or CD16⁺ monocyte frequencies (FIG. 11D), suggesting that NSCLC and melanoma display differences in monocyte profiles associated with aPD-1 response¹⁰.

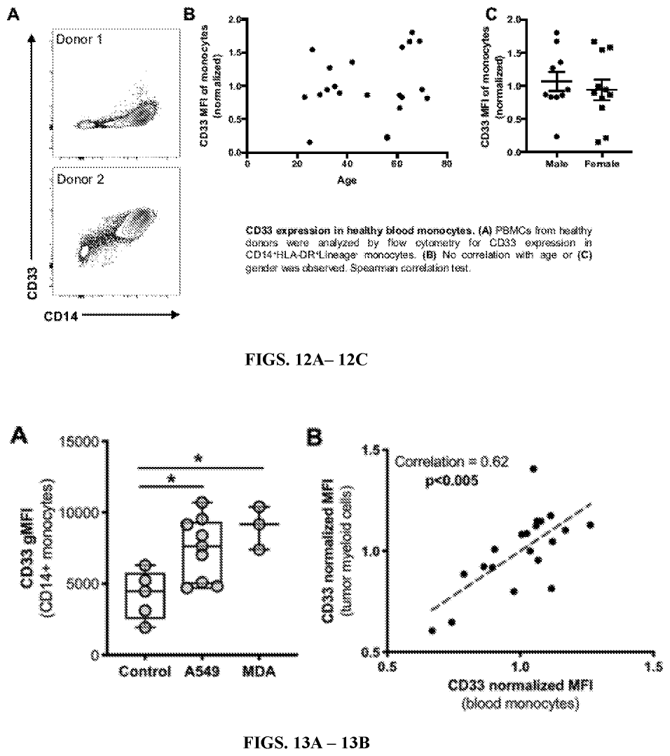

CD33 is a member of the sialic acid-binding membrane proteins called siglecs that are broadly expressed by leukocytes. CD33 is believed to modulate immune responses to self-glycans through signaling through cytoplasmic immunoreceptor tyrosine-based inhibitor motifs (ITIM)¹⁶, which suppresses production of inflammatory cytokines such as IL-1β, TNF-α, and IL-8 in human monocytes¹⁷. Emerging evidence indicates that dysregulation of sialylation is a defining feature in multiple cancer types and is associated with tumor progression^{18,19}. Human monocytes, macrophages, and granulocytes typically express CD33¹⁶, although it has been reported²⁰ that variable CD33 expression in healthy peripheral blood monocytes is not correlated with donor age or gender (FIG. 12). To test cancer-related changes in CD33, sorted human CD14⁺ monocytes were cultured with A549 lung adenocarcinoma and MDA-MB-231 triple-negative breast cancer cell lines. After 12 hours, both cell lines increased CD33 expression in CD14⁺ monocytes (FIG. 13A). It was also observed that that monocytes from melanoma patients have higher CD33 compared to healthy monocytes (data not shown). In re-analysis of published CyTOF data collected from paired PBMCs and tumor biopsies of early lung adenocarcinoma patients¹¹, it was discovered that blood monocyte CD33 expression is highly correlated to CD33 expression by tumor macrophages and DCs (FIG. 13B). Without being bound by theory, these findings suggest that CD33 expression in blood monocytes has implications for signaling within the TIME.

To more mechanistically determine the function of CD33^{hi} monocytes in response to aPD-1 murine CD33 cells were investigated. mouse CD33 lacks ITIM-like domains that human CD33 (hCD33) uses to signal²¹, indicating that murine CD33 is likely unsuitable for studying hCD33 functions. Transgenic Rosa26-Stop^{fl/fl}-hCD33 mice that express the full-length hCD33 protein under control of a loxP-flanked STOP sequence²² were developed These mice were crossed to CX3CR1-Cre mice to generate CX3CR1-Cre^{+/-}R26−hCD33⁺ (CX3CR1^{hCD33/+}) mice that have monocyte-lineage expression of hCD33 in blood (FIG. 14A) and tissues, including lung (data not shown). Given the high expression of monocyte CD33 in responder NSCLC patients, it was hypothesized that hCD33+ monocytes would be anti-tumoral in mice. C57BL/6 mice transplanted with bone marrow from CX3CR1^{hCD33/+} mice were inoculated with subcutaneous (subQ) B16-F10 melanoma tumors (FIG. 14B). Applicants found that tumor size was greater in CX3CR1^{hCD33/+}-transplanted mice compared to controls. In the same mice, MC38 tumors responded to aPD-1 (FIG. 14B) and tumor-bearing hCD33+ mice had higher PD-1 expression in tumor-draining lymph nodes (tdLN) T cells after 13 days, as measured by flow cytometry (FIG. 14C). Without being bound by theory, these findings suggest that CD33 expression in monocytes is pro-tumoral but may prime T cells to be more responsive to aPD-1.

Based on these findings, this disclosure provides methods and treatments that use CD33^{hi} monocytes as a hallmark of productive anti-tumoral immunity that can be targeted to improve ICB responses. These findings are supported by the work reported herein that: 1) CD33^{hi} monocytes are associated with positive clinical response to aPD-1 in advanced NSCLC patients; 2) CD33 expression in the TIME and blood monocytes is highly correlated in early stage NSCLC patients; and 3) Though mice expressing hCD33 have more rapid tumor growth, they display TIME features associated with increased ability to respond to aPD-1.

Lung cancer is the deadliest cancer type worldwide, but whether specific monocyte subsets are important for ICB response in lung cancer has not been explored. This disclosure identifies how monocyte CD33 expression controls anti-tumoral immunity and assesses the therapeutic potential of CD33^{hi} monocytes in increasing aPD-1 efficacy cancer models such as in preclinical lung cancer models. The disclosed methods are based on these reported findings that highlight a role of monocyte-expressed CD33 in positive response to aPD-1 treatment and development of anti-tumoral immunity (FIGS. 11-14).

### Example 12

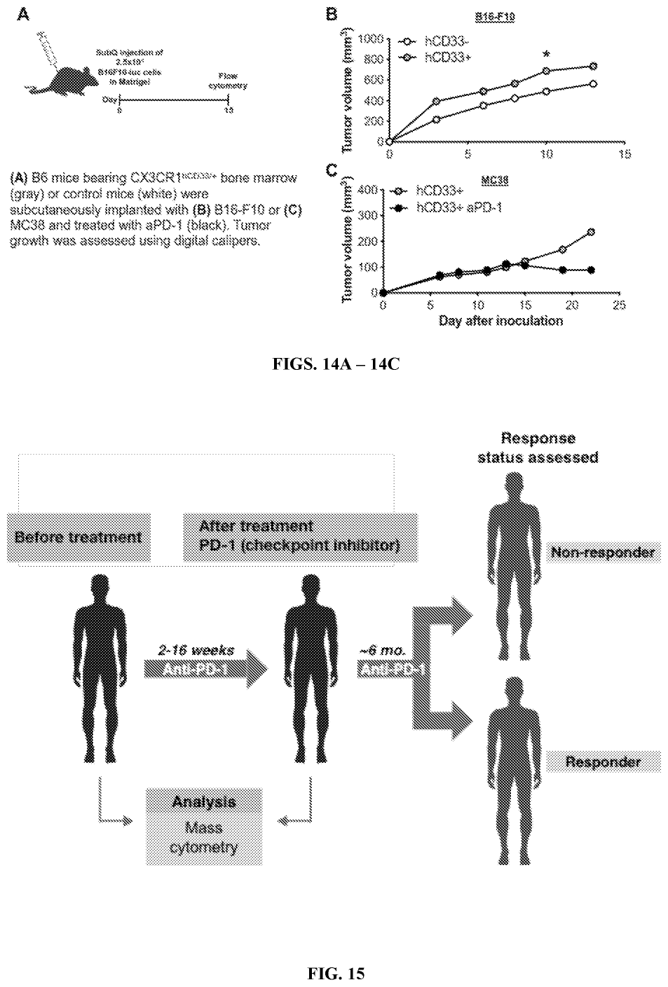

In this example, the applicants demonstrate a non-limiting example of method using a patient cohort used to predict patient response to immunotherapy. The applicants looked to determine whether the phenotype of peripheral blood monocytes can predict whether a patient will respond to anti-PD-1 immunotherapy. Blood was collected from healthy donors and patients with cancer both before and after anti-PD-1 therapy, and the donor was tracked for therapy response. (FIG. 15)

### Example 13

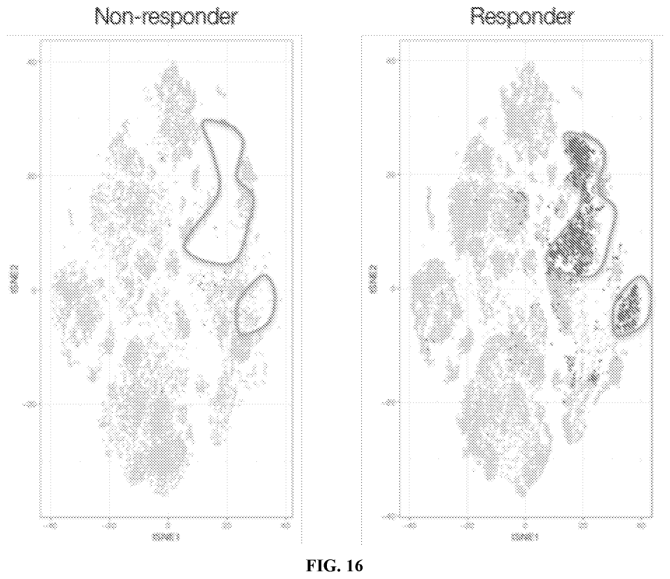

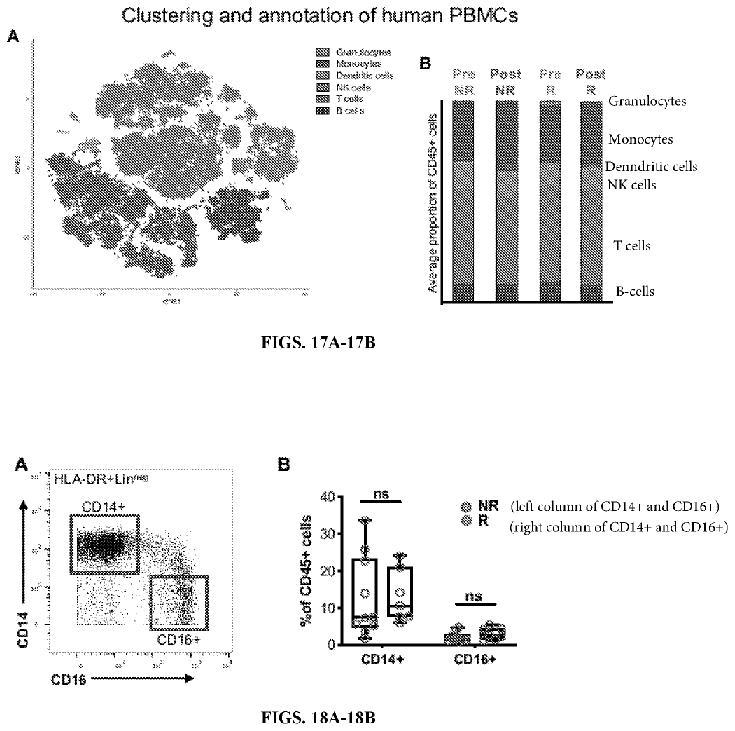

In this example, the applicants demonstrate that two specialized CD33hi monocyte subsets are found in blood of responders to anti-PD-1 immunotherapy (from FIG. 15). CyTOF data was analyzed using t-SNE using machine learning algorithms in an unbiased manner. The circled CD33hi cells are present in responders and absent in non-responder blood (FIG. 16).

### Example 14

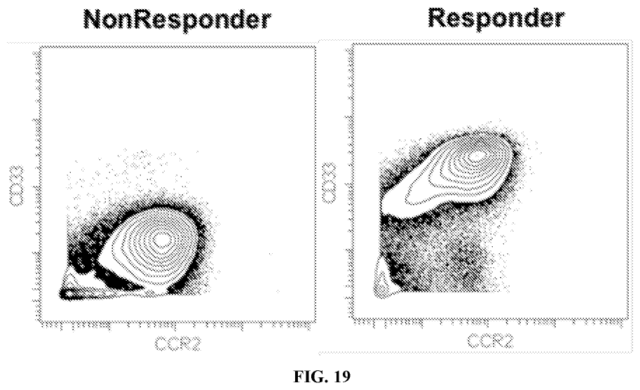

In this example, the applicants demonstrate that CD33hi monocytes are present only in responders. Using biaxial gates identified by GateFinder, Applicants confirmed using conventional flow cytometry that CD33hi monocytes from the cohort of FIG. 15 were only present in responders (FIG. 19).

### Example 15

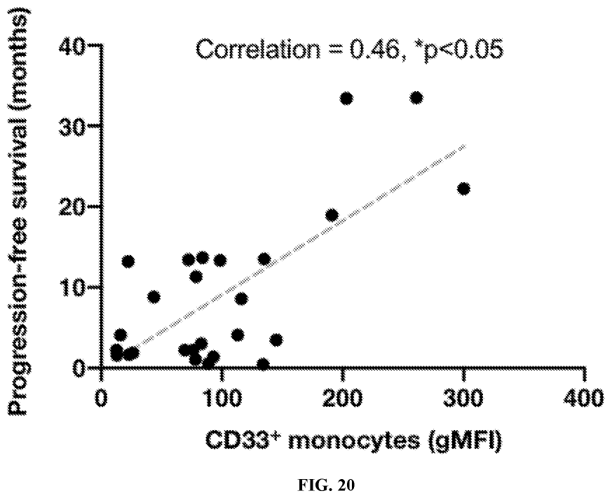

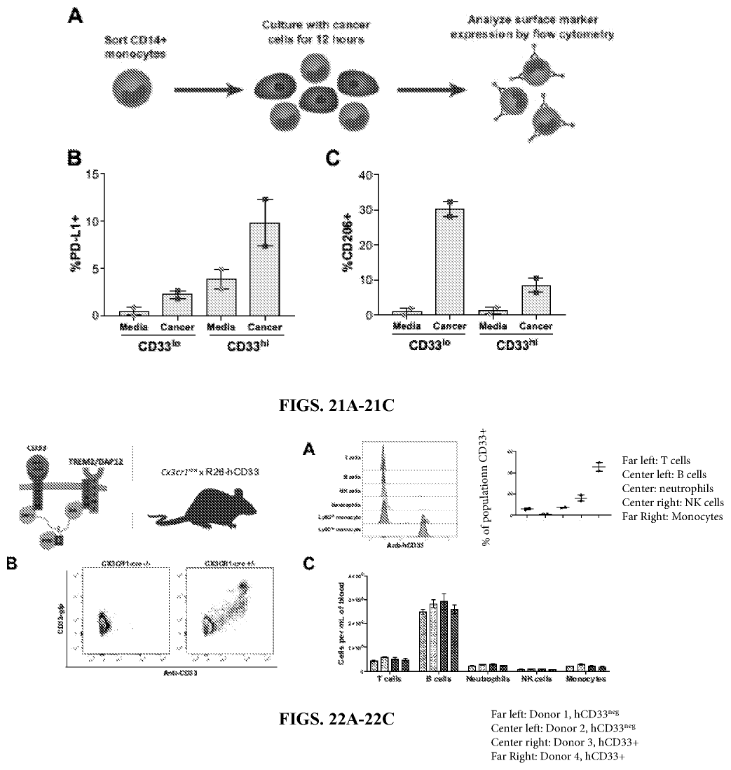

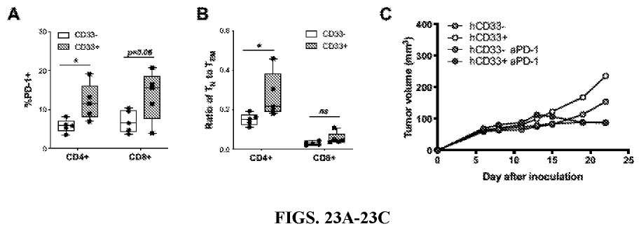

In this example, applicants demonstrate that CD33+ expression on monocytes predicts patient survival (FIG. 20).

Molecular and Anti-Tumoral Properties of CD14+CD33^{hi} Monocytes Associated with Response to ICB in NSCLC Patients.

To further define the function of CD33 expression in CD14 monocytes: (a) single-cell transcriptional profiling can be combined with antibody barcoding to identify transcriptional signatures of CD14t monocytes and T cells associated with CD33 expression in NSCLC patients treated with aPD-1. (b) Identify monocyte features (including CD33 expression) that are linked to clinical outcomes and tumor CD8⁺ T cell infiltration using integrated weighted gene coexpression network analyses. (c) Assess the immuno-modulatory functions of CD14⁺ monocytes from healthy CD33^{hi}/CD33^{lo} donors and aPD-1-treated NSCLC patients.

Sorted blood monocytes can be tested for production of immunomodulatory cytokines in co-culture with human lung adenocarcinoma cells by multiplexed immunoassays and ability to stimulate CD8⁺ T cell activation and proliferation.

Identification of Classical CD33^{hi} Monocytes in the TIME and their Therapeutic Potential in Combination with ICB.

To determine the efficacy of targeting classical CD33 monocytes to improve ICB response in preclinical cancer models, applicants: (a) measure growth of orthotopic lung adenocarcinoma tumors and response to aPD-1 in a novel transgenic mouse that expresses the human CD33 protein in monocyte-lineage cells. (b) Assess the contribution of transferred CD33⁺ monocytes to the TIME by immunophenotyping donor- and host-derived tumor macrophages, DCs, and T cells during aPD-1 treatment. (c) Determine the impact of adoptive CD14⁺CD33^{hi} monocyte therapy on response to aPD-1 in humanized mice bearing patient-derived xenografted lung tumors.

This will identify insight into crosstalk between monocytes and T cells, as well as whether monocyte subsets can be leveraged to enhance response to ICB in lung cancer.

Experimental Details-Molecular and Anti-Tumoral Properties of CD14⁺CD33^{hi} Monocytes Associated with Response to ICB in NSCLC Patients.

Human studies can utilize samples provided by UCSD Moores Cancer Center (MCC) for cancer patient samples, including all frozen PBMCs and tumor sections proposed in this application. Patient samples and data is de-identified. Response status is assessed after 6 months of treatment, with responders defined as patients who showed clinical benefit (decrease in tumor size, reduced occurrence of metastases, or stable disease) for at least the first 6 months of treatment. Non-responders include patients who progressed or discontinued therapy within the first 6 months of treatment.

Applicants hypothesize that CD33^{hi} and CD33^{lo} monocytes from responders and non-responders, respectively, display unique transcriptional features associated with aPD-1 response. Single-cell transcriptional profiling can be combined with oligonucleotide-conjugated antibody barcoding (AbSeq²³) to correlate CD33 expression with transcriptional features of CD14⁺ monocytes and T cells sorted from NSCLC patient blood collected prior to aPD-1 therapy. AbSeq is a commercially available platform (BD Biosciences) that enables parallel and single cell assessment of protein and gene expression levels using predefined panels. AbSeq is particularly well-suited for measurement of low mRNA transcripts (such as CD4 and PD-1) and transcripts that display poor correlation between mRNA and protein, as the inventor observed for CD33. Monocytes (1×10⁴ HLA−DR⁺Lineage⁻ cells) and T cells (1×10⁴ CD3⁺ cells) can be sorted using fluorescence-activated cell sorting (FACS) from 6 non-responder and 6 responder NSCLC patient PBMCs. A comprehensive antibody and gene panel based has been designed on analysis of published single-cell RNA sequencing (scRNA-Seq) and CyTOF data. Sample tagging and pooled antibody staining can be used to minimize batch effects along with BD Rhapsody system to generate single cell mRNA libraries, and a HiSeq2500 for sequencing. AbSeq data can be analyzed using integrated analysis combining the best practice methods for scRNA-Seq and CyTOF, including Seurat²⁴ to normalize any batch effects and clustering (FowSOM, Phenograph), dimensional-reduction (tSNE, UMAP), and differential analysis to identify monocyte and T cell subsets associated with CD33 expression or aPD-1 response, using methods known in the art¹³.

Preliminary data from healthy frozen PMBCs (data not shown) was generated using AbSeq. Based on the inventor finding that monocyte CD33 expression is elevated in NSCLC responders (FIG. 11C), this was expected to be observed in the AbSeq data. It is also expected that genes associated with inflammation, CD8 T cell activation, macrophage/DC differentiation, and cell killing will be co-regulated and elevated in CD33^{hi} monocytes and responders. Alternative results can also be expected as because others have reported T cell changes occur after aPD-1³. Monocytes have already found them to be associated with response (FIG. 11B), and intratumoral T cells.

### Integrated Analysis of Gene Expression Linking CD33^{hi} Monocytes to Clinical Features.

In large transcriptomics datasets, groups of similarly expressed transcripts (gene modules) can be correlated with traits variable across the cohort using weighted gene correlation network analysis (WGCNA)²⁵. This approach was modified (iWGCNA) to form integrated gene modules across patient-matched cell types to reveal molecular crosstalk and relationship to specific traits. Using single cell gene expression (AbSeq) data, iWGCNA is performed to unbiasedly define key monocyte genes that are associated with CD33 expression and NSCLC patient outcomes (response duration, PFS, and overall survival). iWGCNA is also used to determine if monocyte gene signatures are associated with tumor CD8⁺ T cell infiltration. Tumor CD8⁺ cell infiltration is measured by CD8 IHC of formalin-fixed paraffin-embedded tissues (performed by LJI Histology Core). CD8⁺ cell counts and tumor infiltration are assessed in tumor sections from patients with paired PBMCs provided by MCC.

To validate monocyte/T cell gene expression signatures associated with CD33 and response status, a validation cohort of an additional 40 NSCLC patient samples (20 non-responders, 20 responders based on power analysis) is studied using conventional flow cytometry for monocyte markers (HLA-DR, CD14, CD16, and CD33) and any new surface markers identified in AbSeq data. Gene expression modules are validated in sorted monocytes and T cells using standard qRT-PCR²⁶ or nanoString PanCancer Immune Profiling Panel. It is expected that CD33-associated gene modules will be linked to improved clinical outcomes such as longer patient responses and survival, as well as CD8⁺ T cell infiltration. I IHC for macrophage and DC populations that have been investigated in ICB response² can also be performed.

CD14⁺CD33^{hi} and ICB-Associated Monocytes Possess Anti-Tumoral Immune Functions?

CD14+ monocytes are isolated from healthy blood (LJI Clinical Studies Core) for in vitro assessment of cytokine production and T cell activation. Over 30 healthy blood donors were screened for monocyte CD33 expression by flow cytometry (FIG. 12). Individuals with the highest (8 donors) and lowest (8 donors) CD33 expression are selected to serve as CD33^{hi} and CD33^{lo} donors, respectively. To measure production of immunomodulatory cytokines, CD14+ monocytes are isolated from blood using gradient centrifugation and FACs sorting. 1×10⁵ monocytes are added to a transwell insert above A549 cells (1:1 ratio) and cultured for 12 hours, as previously performed and reported (FIG. 13)⁸. Monocyte cell lysates and media are assayed for cytokine production using the LegendPlex ELISA Human Inflammation Panel.

Others have observed variable effects of NSCLC-derived monocytes on T cell activation²⁸, but the source of this heterogeneity has not been explored. To test antigen non-specific T cell activation, CD14⁺ monocytes and T cells are sorted from the indicated CD33^{hi}/CD33^{lo} donors or NSCLC patients (4 non-responders, 4 responders). CellTrace Violet-labeled T cells are combined with 1×10⁵ CD14⁺ monocytes at a 2:1 ratio in the presence of anti-CD3 for 24 hours, using methods known in the art and reported²⁹. Flow cytometry is used to measure production of effector cytokines (IFN-γ, TNF-α, IL-2, IL-6, Granzyme B, and Perforin), proliferation via CellTrace Violet dilution, and surface markers (CD4, CD8, CD45RA, CD45RO, CCR7, CD25, PD-1, and CD69). T cells stimulated with CD3/CD28 dynabeads are used as a positive control and unstimulated T cells as a negative control.

As CD33^{hi} monocytes are elevated in responder NSCLC patients prior to the start of aPD-1, CD33^{hi} monocytes from healthy donors may display increased anti-tumoral immune functions relative to CD33^{lo} monocytes, including increased secretion of pro-inflammatory cytokines (IL-1β, TNF-α, and IL-12) and stimulation of T cell proliferation/activation. T cells cultured with CD14⁺ monocytes from NSCLC responders may synthesize increased effector cytokines and upregulate surface markers associated with activation. Without being bound by theory, most effects are likely to occur in CD8⁺ T cells given their documented role in response to aPD-1³. Differences in cytokine production/T cell activation in healthy monocytes can also be monitored and observed if they are inherently different from NSCLC patients. Tumor cell cytotoxicity and differentiation into pro-inflammatory macrophage/DCs can also be tested.

Classical CD33hi Monocytes in the TIME and Define their Therapeutic Potential in Combination with ICB.

Contribution of human CD33 expression in monocyte lineage cells to response to aPD-1. Although hCD33 promotes tumor growth in mice (FIG. 14B), it was observed that mice with hCD33⁺ monocytes are responsive to aPD-1 and have more PD-1⁺ T cells (FIG. 14B, 14C). In order to specifically explore the function of monocyte hCD33 in a preclinical model of lung cancer, syngeneic CMT167 lung adenocarcinoma tumors are orthotopically implanted in the lungs of CX3CR1^{hCD33/+} mice. Importantly, aPD-1 treatment promotes regression of orthotopic CMT167 tumors³⁰. CMT167 cells are stably transfected with firefly luciferase to enable monitoring of tumor growth using an IVIS Bioluminescence Imager and digital calipers. 2.5×10⁵ CMT167 cells are injected into the left lung using methods known in the art⁸. Tumor growth is measured in CX3CR1^{hCD33/+} mice and CX3CR1-Cre⁻R26-hCD33⁺ control mice. Half of the mice receive aPD-1 (RMP1-14, 200 g per mouse per injection) or isotype control (IgG2a) intraperitoneally at 8, 11, and 14 days after tumor inoculation, a dosing regimen that produces regression of aPD-1-responsive tumors (FIG. 14B).

Without being bound by theory, hCD33 expression is likely to have a synergistic effect with aPD-1 on tumor growth and regression, which will be observed if CX3CR1^{hCD33/+} treated with aPD-1 display significantly smaller tumors compared to both untreated CX3CR1^{hCD33/+} and control mice. Decreasing doses of aPD-1 can be administered to produce a sub-optimal therapeutic response alone and/or tested in another lung cancer cell line such as Lewis lung carcinoma (LLC1) s and is less responsive to aPD-1³⁰. If CMT167 tumors are slow growers, the model can be modified by delaying aPD-1 administration until tumor size reaches 50-100 mm³.

Impact of Monocyte-Expressed hCD33 on the TIME.

It is shown herein that CD33^{hi} monocytes contribute to a pro-tumoral TIME during tumor progression (FIG. 14), but acutely adopt fates during aPD-1 treatment that support effective tumor immunity. In order to determine the impact of hCD33 on monocyte fate and the TIME during aPD-1 treatment, hCD33+ monocytes are adoptively transferred during early lung tumor establishment (day 3) and one day prior to aPD-1 (day 7). 5×10⁵ monocytes (CD115⁺CD11b⁺) sorted from blood/spleens of CX3CR^{hCD33/+} or control mice are transferred into congenically marked CD45.1 mice bearing CMT167 tumors. Tumors are explanted and digested at 12 days (after aPD-1, but prior to large differences in tumor size) for quantification of host (CD45.1⁺) and donor-derived tumor macrophages (F4/80^{hi}CD24^{lo}CD11b⁺Siglec-F⁻), CD103⁺ and CD11b⁺ DCs (F4/80^{lo}CD24^{hi} MHCII^{hi})³¹, and T cells (CD3⁺ TCRβ⁻). Tumor macrophages/DCs are further characterized based on expression of markers for inflammatory (MHCII, iNOS, CD86) and immunosuppressive (CD206, Arginase, PD-L1, PD-L2) phenotypes. Tumor and tdLN T cells can be discriminated into CD4⁺ and CD8⁺ naïve and effector populations based on CD44/CD62L expression and Foxp3⁺ regulatory T cells. T cells are also be assessed for LAG3, CD69, and CXCR3 expression.

Given the increased tumor growth observed in CX3CR1^{hCD33/+} mice (FIG. 14B), it is expected that macrophage/DC progeny derived from hCD33⁺ monocytes transferred 3 days post tumor inoculation will display a more immunosuppressive phenotype compared to hCD33-control monocytes and monocytes transferred 1 day prior to aPD-1 treatment. Conversely, progeny derived from hCD33⁺ monocytes transferred 1 day prior to aPD-1 are expected to display an inflammatory phenotype compared to hCD33⁻ monocytes transferred at the same time and this will be associated with more activated CD4⁺/CD8⁺ T cells (higher expression of CD44, CD69, and CXCR3 activation markers; reduced expression of LAG3³⁴) in tumors and tdLN.

Does CD14⁺CD33hi Monocyte Transfer Increase Therapeutic Response to aPD-1?

Without being bound by theory, it is expected that CD33^{hi} monocytes promote productive anti-tumoral immune responses and lung tumor rejection during aPD-1 treatment, making them an attractive approach for combination therapy. Early phase clinical trials are exploring adoptive monocyte therapy for treatment of solid tumors³⁵, indicating that monocyte-based cell therapies have potential for clinical translation. Transferred CD14⁺CD33^{hi} monocytes are transferred to increase therapeutic response to aPD-1 in a humanized mouse bearing patient-derived xenograft (PDX) lung tumors. Immunodeficient NSG-SGM3 mice engrafted with human CD34⁺ cells (Hu-NSGM3 mice) are commercially available (The Jackson Laboratory) and support engraftment of non-HLA matched PDX tumors. 1×10⁶ TM00302 cells (a metastatic lung adenocarcinoma PDX) are implanted subQ in the rear flank of Hu-NSGM3 mice. When tumors reach 100 mm³, Hu-NSGM3 are treated with human aPD-1 (5 mg/kg i.p. every 5 days, 5 total doses), as indicated in data provided by The Jackson Laboratory. This dosing significantly reduces tumor size but does not support complete regression. To model the human findings that elevated CD14⁺CD33^{hi} monocytesprior to aPD-1 therapy are associated with a positive clinical response (FIG. 11C), 5×10⁵ CD14⁺CD33^{hi} or CD14⁺CD33^{lo} monocytes sorted from fresh human blood are transferred 1 day prior to aPD-1. Tumor size is measured with a digital caliper by laboratory personnel blinded to the study.

Without being bound by theory transfer of CD14⁺CD33^{hi} monocytes in aPD-1-treated Hu-NSGM3 will reduce tumor size compared to mice receiving aPD-1 alone or CD14⁺CD33^{lo} monocytes. It is possible that there will be reduced tumor growth after transfer CD14⁺CD33^{lo} monocytes, as total CD14⁺ cell counts are associated with positive response to aPD-1 in melanoma patients^{lo}. Dosing can be repeated to optimize the response. Maximal response in this animal model is observed at 21 days post-aPD-1, so this will leave a window for further intervention.

Other examples of implementations will become apparent to the reader in view of the teachings of the present description and as such, will not be further described here.

Note that titles or subtitles may be used throughout the present disclosure for convenience of a reader, but in no way should these limit the scope of the disclosure. Moreover, certain theories may be proposed and disclosed herein; however, in no way they, whether they are right or wrong, should limit the scope of the disclosure so long as the disclosure is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this disclosure pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

With respect to ranges of values, the disclosure encompasses the upper and lower limits and each intervening value between the upper and lower limits of the range to at least a tenth of the upper and lower limit's unit, unless the context clearly indicates otherwise. Further, the disclosure encompasses any other stated intervening values.

Although various embodiments of the disclosure have been described and illustrated, it will be apparent to those skilled in the art in light of the present description that numerous modifications and variations can be made. The scope of the disclosure is defined more particularly in the appended claims.

### REFERENCES

1. Bray, F. et al. Global cancer statistics 2018: GLOBOCAN estimates of incidence and mortality worldwide for 36 cancers in 185 countries. CA Cancer J Clin 68, 394-424 (2018).
2. Malhotra, J., Jabbour, S. K. & Aisner, J. Current state of immunotherapy for non-small cell lung cancer. Transl Lung Cancer Res 6, 196-211 (2017).
3. Huang, A. C. et al. T-cell invigoration to tumour burden ratio associated with anti-PD-1 response. Nature 2014 515:7528 545, 60-65 (2017).
4. Olingy, C. E., Dinh, H. Q. & Hedrick, C. C. Monocyte heterogeneity and functions in cancer. Journal of Leukocyte Biology 11, 762 (2019).
5. Spitzer, M. H. et al. Systemic Immunity Is Required for Effective Cancer Immunotherapy. Cell 168, 487-502.e15 (2017).
6. Riaz, N. et al. Tumor and Microenvironment Evolution during Immunotherapy with Nivolumab. Cell 171, 934-949.e16 (2017).
7. Qian, B.-Z. et al. CCL2 recruits inflammatory monocytes to facilitate breast-tumour metastasis. Nature 2014 515:7528 475, 222-225 (2011).
8. Hanna, R. N. et al. Patrolling monocytes control tumor metastasis to the lung. Science 350, 985-990(2015).
9. Franklin, R. A. et al. The cellular and molecular origin of tumor-associated macrophages. Science 344, 921-925 (2014).
10. Krieg, C. et al. High-dimensional single-cell analysis predicts response to anti-PD-1 immunotherapy. Nature Medicine 2018 9, 2579 (2018).
11. Lavin, Y. et al. Innate Immune Landscape in Early Lung Adenocarcinoma by Paired Single-Cell Analyses. Cell 169, 750-765.e17 (2017).
12. Cannarile, M. A. et al. Colony-stimulating factor 1 receptor (CSF1R) inhibitors in cancer therapy. j. immunotherapy cancer 5, 705 (2017).
13. Hamers, A. A. J. et al. Human Monocyte Heterogeneity as Revealed by High-Dimensional Mass Cytometry. Arterioscler. Thromb. Vasc. Biol. 39, 25-36 (2019).
14. Villani, A.-C. et al. Single-cell RNA-seq reveals new types of human blood dendritic cells, monocytes, and progenitors. Science 356, eaah4573 (2017).
15. Arvaniti, E. & Claassen, M. Sensitive detection of rare disease-associated cell subsets via representation learning. Nature Communications 2017 8:18, 14825 (2017).
16. Macauley, M. S., Crocker, P. R. & Paulson, J. C. Siglec-mediated regulation of immune cell function in disease. Nature Reviews Immunology 2018 14, 653-666 (2014).
17. Lajaunias, F., Dayer, J.-M. & Chizzolini, C. Constitutive repressor activity of CD33 on human monocytes requires sialic acid recognition and phosphoinositide 3-kinase-mediated intracellular signaling. Eur. J. Immunol. 35, 243-251 (2005).
18. Julien, S. et al. Selectin Ligand Sialyl-Lewis x Antigen Drives Metastasis of Hormone-Dependent Breast Cancers. Cancer Res 71, 7683-7693 (2011).
19. Bull, C., Stoel, M. A., Brok, den, M. H. & Adema, G. J. Sialic Acids Sweeten a Tumor's Life. Cancer Res 74, 3199-3204 (2014).
20. Bradshaw, E. M. et al. <i>CD33</i> Alzheimer's disease locus: altered monocyte function and amyloid biology. Nature Neuroscience 2013 16:7 16, 848-850 (2013).
21. Cao, H. & Crocker, P. R. Evolution of CD33-related siglecs: regulating host immune functions and escaping pathogen exploitation? Immunology 132, 18-26 (2010).
22. Duan, S. et al. CD33 recruitment inhibits IgE-mediated anaphylaxis and desensitizes mast cells to allergen. J Clin Invest 129, 1387-1401 (2019).
23. Shahi, P., Kim, S. C., Haliburton, J. R., Gartner, Z. J. & Abate, A. R. Abseq: Ultrahigh-throughput single cell protein profiling with droplet microfluidic barcoding. Scientific Reports 2016 6 7, 44447 (2017).
24. Butler, A., Hoffman, P., Smibert, P., Papalexi, E. & Satija, R. Integrating single-cell transcriptomic data across different conditions, technologies, and species. Nature Biotechnology 201129:1136, 411-420 (2018).
25. Langfelder, P. & Horvath, S. WGCNA: an R package for weighted correlation network analysis. BMC Bioinformatics 9, 1 (2008).
26. Thomas, G. D. et al. Deleting an Nr4a1 Super-Enhancer Subdomain Ablates Ly6Clow Monocytes while Preserving Macrophage Gene Function. Immunity 45, 975-987 (2016).
27. Barry, K. C. et al. A natural killer-dendritic cell axis defines checkpoint therapy-responsive tumor microenvironments. Nature Medicine 2018 24, 1178-1191 (2018).
28. Singhal, S. et al. Human tumor-associated monocytes/macrophages and their regulation of T cell responses in early-stage lung cancer. Science Translational Medicine 11, eaat1500 (2019).
29. Zhu, Y. P. et al. Identification of an Early Unipotent Neutrophil Progenitor with Pro-tumoral Activity in Mouse and Human Bone Marrow. Cell Reports 24, 2329-2341.e8 (2018).
30. Li, H. Y. et al. The Tumor Microenvironment Regulates Sensitivity of Murine Lung Tumors to PD-1/PD-L1 Antibody Blockade. Cancer Immunol Res 5, 767-777 (2017).
31. Broz, M. L. et al. Dissecting the Tumor Myeloid Compartment Reveals Rare Activating Antigen-Presenting Cells Critical for T Cell Immunity. Cancer Cell 26, 638-652 (2014).
32. Olingy, C. E. et al. Non-classical monocytes are biased progenitors of wound healing macrophages during soft tissue injury. Scientifc Reports 2016 6 7, 447 (2017).
33. Loyher, P.-L. et al. Macrophages of distinct origins contribute to tumor development in the lung. Journal of Experimental Medicine 87, jem.20180534 (2018).
34. Blackburn, S. D. et al. Coregulation of CD8<sup>+</sup> T cell exhaustion by multiple inhibitory receptors during chronic viral infection. Nature Immunology 201112:8 10, 29-37 (2009).
35. Green, D. S. et al. A Phase 1 trial of autologous monocytes stimulated ex vivo with Sylatron® (Peginterferon alfa-2b) and Actimmune® (Interferon gamma-1b) for intra-peritoneal administration in recurrent ovarian cancer. Journal of Translational Medicine 2018 16:116, 196 (2018).

What is claimed:

1. A method of determining whether a subject will respond to a treatment for a neoplasia, neoplastic disorder, tumor, cancer or malignancy, the method comprising detecting the amount of CD33 in a biological sample from the subject, comparing the measured amount to a reference amount, wherein a modified measured amount compared to the reference amount is indicative that the subject will or will not respond to the treatment for a neoplasia, neoplastic disorder, tumor, cancer or malignancy.

2. The method of claim 1, wherein the modified measured amount is an increase in the measured amount.

3. The method of claim 1, wherein the modified measured amount is a decrease in the measured amount.

4. The method of claim 2, wherein the increase in the measured amount is indicative that the subject will respond to the treatment for a neoplasia, neoplastic disorder, tumor, cancer or malignancy.

5. The method of claim 3, wherein the decrease in the measured amount compared to the reference amount is indicative that the subject will not respond to the treatment for a neoplasia, neoplastic disorder, tumor, cancer or malignancy.

6. The method of claim 2 or 4, wherein the measured amount is CD33 high expression in the biological sample.

7. The method of claim 3 or 5, wherein the measured amount is CD33 low expression in the biological sample.

8. The method of claim 2 or 4, wherein the measured amount is CD33 low expression in the biological sample.

9. The method of claim 3 or 5, wherein the measured amount is CD33 high expression in the biological sample.

10. A method of determining whether a subject will respond to a treatment for a neoplasia, neoplastic disorder, tumor, cancer or malignancy, the method comprising detecting the presence or amount of CD33 expressing cells in a biological sample from the subject, comparing the measured presence or amount to a reference presence or amount, wherein a modified measured presence or amount as compared to the reference presence or amount is indicative that the subject will or will not respond to the treatment for a neoplasia, neoplastic disorder, tumor, cancer or malignancy.

11. The method of claim 10, wherein the CD33 expressing cells measured are cells with high CD33 expression.

12. The method of claim 10, wherein the CD33 expressing cells measured are cells with low CD33 expression.

13. The method of claim 11, wherein the measured presence or amount of cells with high CD33 expression is indicative that the subject will respond to the treatment for a neoplasia, neoplastic disorder, tumor, cancer or malignancy.

14. The method of claim 12, wherein the measured presence or amount of cells with low CD33 expression is indicative that the subject will not respond to the treatment for a neoplasia, neoplastic disorder, tumor, cancer or malignancy.

15. A method of determining whether a subject will respond to a treatment for a neoplasia, neoplastic disorder, tumor, cancer or malignancy, the method comprising detecting the presence or measuring the amount of cells with high expression of CD33 in a biological sample from the subject, wherein the presence of cells with high expression of CD33 or a high amount of cells with high expression of CD33 indicates that the subject will respond to the treatment for a neoplasia, neoplastic disorder, tumor, cancer or malignancy.

16. The method of claim 15, wherein the method comprises detecting the presence of or measuring the amount of CD33^{hi} myeloid cells in the biological sample.

17. The method of any preceding claim, wherein the method comprises detecting the presence or measuring the amount of classical or non-classical monocytes in the biological sample.

18. The method of claim 17, wherein the method comprises detecting the presence or measuring the amount of classical monocytes in the biological sample.

19. The method of claim 17, wherein the method comprises detecting the presence or measuring the amount of non-classical monocytes in the biological sample.

20. The method of any one of claims 6, 8, 11, 13, and 15, wherein the CD33 high expressing cells comprise CD33 monocytes or CD33^{hi} macrophages in the biological sample.

21. The method of claim 20, wherein the method comprises detecting or measuring the amount of CD33^{hi}CD19−CD3−CD66b−CD56− cells or CD33^{hi}CD3−CD19−CD66b−CD56−HLA−DR+CD86⁺ cells in the biological sample.

22. The method of claim 20, wherein the method comprises detecting or measuring the amount of CD33^{hi} CD14^{dim}CD16⁺ monocytes or macrophages, CD33^{hi}CD14⁺CD16^{lo} monocytes or macrophages, CD33^{hi}CD14^{hi}CD16⁻ monocytes or macrophages, intermediate CD33^{hi}CD14⁺CD16⁺ monocytes or macrophages, or CD33^{hi}CD14^{lo}CD16⁺ monocytes or macrophages in the biological sample.

23. A method of determining whether a subject will respond to a treatment for a neoplasia, neoplastic disorder, tumor, cancer or malignancy, the method comprising detecting the presence or measuring the amount of cells with low expression of CD33 in a biological sample from the subject, comparing the presence or measured amount to a reference amount, wherein a decreased presence or measured amount compared to the reference amount is indicative that the subject will not respond to the treatment for a neoplasia, neoplastic disorder, tumor, cancer or malignancy.

24. The method of claim 23, wherein the method comprises detecting the presence or measuring the amount of CD33^{lo} myeloid cells in the biological sample.

25. The method of claim 23 or 24, wherein the method comprises detecting the presence or measuring the amount of classical or non-classical monocytes in the biological sample.

26. The method of claim 25, wherein the method comprises detecting the presence or measuring the amount of classical monocytes in the biological sample.

27. The method of claim 25, wherein the method comprises detecting the presence or measuring the amount of non-classical monocytes in the biological sample.

28. The method of claim 23, wherein the method comprises detecting the presence of or measuring the amount of CD33^{lo} monocytes or CD33^{lo} macrophages in the biological sample.

29. The method of claim 28, wherein the method comprises detecting the presence of or amount of CD33^{lo}CD19−CD3−CD66b−CD56− cells or CD33^{lo}CD3−CD19−CD66b−CD56−HLA−DR+CD86⁺ cells in the biological sample.

30. The method of claim 28, wherein the method comprises detecting the presence of or amount of CD33^{lo}CD14^{lo}CD16⁺ monocytes or macrophages, CD33^{lo}CD14⁺CD16^{lo} monocytes or macrophages, CD33^{lo}CD14^{hi}CD16⁻ monocytes or macrophages, intermediate CD33^{lo}CD14⁺CD16⁺ monocytes or macrophages, or CD33^{lo}CD14^{lo}CD16⁺ monocytes or macrophages in the biological sample.

31. The method of any preceding claim, wherein the subject has a neoplasia, neoplastic disorder, tumor, cancer or malignancy.

32. The method of claim 31, wherein the cancer is a carcinoma or a leukemia.

33. The method of claim 31, wherein the subject has non-small cell lung cancer, head and neck squamous cell carcinoma, breast cancer, or melanoma.

34. The method of any preceding claim, wherein the subject is suffering from a cancer of the group of: Stage I, Stage II, Stage III or Stage IV.

35. The method of any preceding claim, wherein the biological sample comprises a blood sample.

36. The method of any preceding claim, wherein the biological sample is taken from the subject prior to the treatment.

37. The method of any preceding claim, further comprising taking a further biological sample from the subject after treatment to monitor treatment efficacy.

38. The method of any preceding claim, wherein a positive response to treatment for the neoplasia, neoplastic disorder, tumor, cancer or malignancy comprises one or more of an increase in survival time; an increase in elongation in time to tumor progression; a reduction in tumor mass; a reduction in tumor burden and/or a prolongation in time to tumor metastasis; elongated time to tumor recurrence, tumor response, complete response, partial response, stable disease, progressive disease; an increase in progression free survival, or an increase in overall survival.

39. The method of any preceding claim, wherein the treatment is selected from the group of: a first line treatment, a second line treatment, a third line treatment or a fourth line treatment.

40. The method of any preceding claim, wherein the treatment comprises or consists essentially of a check point blocker.

41. The method of claim 39, wherein the treatment comprises or consists essentially of a PD-1 inhibitor or a PD-L1 inhibitor.

42. The method of claim 40, wherein the check point blocker is selected from the group of: an anti-PDL-1 antibody, an anti-PD-1 antibody, Nivolumab, Pembrolizumab, Cemiplimab, Atezolizumab, Avelumab, Darvalumab, or an equivalent of each thereof.

43. The method of claim 41, wherein the PD-1 inhibitor is selected from the group of an anti-PD-1 antibody, Nivolumab, Pembrolizumab, Cemiplimab, or an equivalent of each thereof.

44. The method of claim 41, wherein the PD-L1 inhibitor is selected from the group of Atezolizumab, Avelumab, Darvalumab, or an equivalent of each thereof.

45. The method of claim 43, wherein the cancer is from the group of: melanoma, squamous non-small cell lung cancer, renal cell carcinoma, myeloma, cutaneous squamous cell carcinoma, bladder cancer, Hodgkin's lymphoma, a unresectable or metastatic solid tumor, or small cell lung cancer.

46. The method of claim 44, wherein the cancer is from the group of: bladder cancer, non-small cell lung cancer, small cell lung cancer, or Merkel-cell carcinoma.

47. The method of any preceding claim, further comprising administering an effective amount of the treatment to the subject where the subject has been determined to respond to the treatment.

48. The method of any one of claims 1-46, further comprising administering an effective amount of an expanded population of CD33^{high} expressing cells to the subject where the subject has been determined to not to respond to the treatment.

49. The method of claim 48, wherein the CD33^{high} expressing cells comprise CD33^{hi}CD19−CD3−CD66b−CD56− cells or CD33^{hi}CD3−CD19−CD66b−CD56-HLA-DR+CD86+ cells.

50. The method of claim 48, wherein the CD33^{high} expressing cells comprise CD33^{hi}CD14^{dim}CD16⁺ monocytes or macrophages, CD33^{hi}CD14⁺CD16^{lo} monocytes or macrophages, intermediate CD33^{hi}CD14⁺CD16⁺ monocytes or macrophages, CD33^{hi}CD14^{hi}CD16⁻ monocytes or macrophages, CD33^{lo}CD14^{hi}CD16⁻ monocytes or macrophages, and CD33^{hi}CD14^{lo}CD16⁺ monocyte or macrophages.

51. The method of any one of claims 48-50, further comprising administering an effective amount of the treatment to the subject.

52. The method of any one of claims 47-51, wherein the treatment is selected from the group of: a first line treatment, a second line treatment, a third line treatment or a fourth line treatment.

53. The method of any one of claims 47-52, wherein the treatment is co-administered with an effective amount of a second therapy.

54. The method of claim 53, wherein the second therapy is administered prior to, concurrently or subsequently to the treatment.

55. The method of claim 53, wherein the second therapy comprises one or more of surgical rescission, radiation therapy, light therapy, or a chemotherapy.

56. The method of any preceding claim, wherein the subject is a mammal.

57. The method of claim 56, wherein the mammal is of the group of: a canine, a feline, an equine, a bovine, an ovine, a murine, a rat, a simian or a human patient.

58. The method of any preceding claim, wherein the subject is a female.

59. The method of any preceding claim, wherein the subject is a male.

60. The method of any preceding claim, wherein the subject is a pediatric patient.

61. The method of claim 53, wherein the effective amount of the second therapy is a low dose therapy.

62. The method of claim 37, further comprising administering an effective amount of the same treatment or different therapy to the subject.

63. The method of claim 62, wherein administering an effective amount of the same treatment to the subject comprises administering a modified effective amount of the treatment.

64. A kit comprising reagents to detect or measure the presence or amount of CD33 or CD33 expressing cells in a biological sample and instructions for use in the methods of claims 1 to 63.

65. A kit comprising reagents to detect or measure the amount of CD33^{hi} and/or CD³³low expressing cells in a biological sample and instructions for use in the methods of claim 1 to 63.

66. The method of any one of claims 1 to 63, wherein the detecting or measuring is by a method comprising one or more of DNA microarrays, Real-time PCR, Chromatin immunoprecipitation (ChIP), flow cytometry, Western blotting, 2-D gel electrophoresis, immunoassays, or Fluorescence-activated cell sorting.

67. A method of treating a neoplasia, neoplastic disorder, tumor, cancer or malignancy in a subject, the method comprising modulating CD33 activity or expression in a myeloid cell.

68. The method of claim 67, wherein the method comprises modulating up CD33 activity or expression in a myeloid cell.

69. The method of claim 67, wherein the method comprises modulating down CD33 activity or expression in a myeloid cell.

70. The method of any of claims 67-69, wherein the method comprises modulating the amount of one or more of: CD33hiCD19−CD3−CD66b−CD56− cells, CD33hiCD3−CD19−CD66b−CD56−HLA-DR+CD86+ cells, CD33hi CD14dimCD16+ monocyte or macrophages, CD33hiCD14+CD16lo monocyte or macrophages, intermediate CD33hiCD14⁺CD16⁺ monocyte or macrophages, and/or CD33hiCD14loCD16+ monocyte or macrophages in the biological sample.

71. The method of any of claims 67-69, wherein the method comprises modulating the amount of one or more of: CD33loCD19−CD3−CD66b−CD56− cells, CD33loCD3-CD19−CD66b−CD56-HLA-DR+CD86+ cells, CD33loCD14dimCD16+ monocyte or macrophages, CD33loCD14+CD16lo monocyte or macrophages, intermediate CD33loCD14+CD16+ monocyte or macrophages, and/or CD33loCD14loCD16+ monocyte or macrophages in the biological sample.

72. The method of claim 67, wherein the method comprises administering an agent that modulates CD33 activity or expression.

73. The method of claim 72, wherein the agent is a small molecule, an antibody, a protein or a peptide.

74. The method of claim 72, wherein the agent is azacitidine, decitabine, or lintuzumab.

75. A method of treating a neoplasia, neoplastic disorder, tumor, cancer or malignancy in a subject, the method comprising administering a population of CD33hi monocytes or CD33hi macrophages.

76. The method of claim 75, wherein the CD33hi monocyte or macrophage population comprises one or more of: CD33hiCD19−CD3−CD66b−CD56− cells, CD33hiCD3−CD19−CD66b−CD56−HLA-DR+CD86+ cells, CD33hi CD14dimCD16+ monocyte or macrophages, CD33hiCD14+CD16lo monocyte or macrophages, intermediate CD33hiCD14+CD16+ monocyte or macrophages, and/or CD33hiCD14loCD16+ monocyte or macrophages in the biological sample.
